SEQUENCE LISTING (21 sequences)

SEQ ID NO: 1 - DNA, 1587 bp, Unknown
  Description of sequence sequence from genomic DNA library from rumen ecosystem, particularly nucleic acid sequence of clones pBKRR 1 and 21; RR01-1 First enzyme (= RR21); CDS; (see Figure 9)
atgaaactgtattggaaatatctttcattgactgctgtgctggctgtcctggcttcctgcggcggcaatgctgatcccgatccggagccgacgccctcggcgccatcatccattaccttgagcgtcaattcctttacagttgcgcaggccggtgagaccctgtccctggacattacggctcccactcgtccgaaactcgcactcccgaactggattactctcaaggacggtacctatcagaattataagattaccgtgggactgacggtttcggccaatgatacgtaccaggcacgtgaggcgactgtgacggtttctgccacggggactttcgacgttaccttcaaggtttcccaggccgggaaagaacctgaacccacgccgccggatccgagcggcggggacaacgatgcctggcagatggcgaagaagttggggttgggctggaacatgggcaatcatttcgatgctttctacaatggttcctgggccggtgagaaggaaggctatccggacgaggaggtctggggtgcgtcgaaggctacgcagacgacattcaatggcgtgaagaatgccggctttacgagtgtccgcatccctgtctcctggctgaagatgatcggtccggctccggaatacaagattgacgagacttggctgaaccgcgtgtatgaggttgcgcagtatgcccataatgccggactgactgcgattgtcaatacgcaccatgatgagaaccacggcgtggacaatacctatcagtggctggacatcaagaatgcttcgaccgacgcttccgtgaatacgcggatcaaggaggaaatcaaggccgtctggacccagatcgcgaacaagttcaaggattgtggagactggctcatcctggagagtttcaacgaattgaacgacggtggctggggctggagcgatgctttccgggccaatccgaccaaacagtgcaatatcctgaacgagtggaaccaggtattcgtggatgcggtccgtgccaccggcgggaacaatgccacccgctggctgggcgtcccgacgtatgcggccaatccggagttcgagaaatatgcgactcttccgaccgacagtgccaacaagatcatgctggcggtacatttctacgatccgtcggactataccatcggcgatgcgcagtacagcgactgggggcataccggtgcggctgacaagaaggcctcgggtggggacgaggaccatgtccgttccgtcttcggcaacctgtgcacgaagtatgtggaaaataacatcccggtctatctgggcgaattcggctgctccatgcgggcgaaatccaatacgcgtgcctggaaattctatctgtattacatggagtatatcgtcaaggcagccaagacctacggccttccttgttatctttgggacaatggctccaagagtgagcccggtaaggaagtccatggctatatagaccatggaacaggtgactatatcggcaatagcaaggaagtaatcgatgtcatgaagaaagcgtggtttaccgagtcggcaggttataccctccagacggtatataatgctgcgccgaaattgtaa

SEQ ID NO: 2 - DNA, 1152 bp, Unknown
  Description of sequence sequence from genomic DNA library from rumen ecosystem, particularly nucleic acid sequence of clones pBKRR 1 and 21; RR01-2 Second enzyme (= RR21); CDS; (see Figure 9)
atgctgcgccgaaattgtaatcatctgaatatgaaacatatagctttatattttatgctgggagccgttgccctggccgccccgggctgcggccggcgggctgaaagacccgctacgaccccgaaggaccaggtgatcgcgcaactgcaggaagccgtgaacggcggcaagattctctatgcccaccaggacgacctggtgtatggccacacctggaaagtagaagatgtggcgggtgatccgctggagcgttccgacgtcaaggccgtgacaggattctatccggcgatggtcggcttcgacctgggcggcatcgagatgggctgggaggccaatctggacggcgtcccgttcgacctgatgcgccgggcggccgtgacccacgcctcccgcggcggcatcgtgaccttctcctggcatccgcgcaatccgttgatcggcggagacgcctgggatgtctcttccgaccaggtcgtcgcttccatcctcgaaggaggggagaagcatgacctgttcatggaatggctcaggcgtgcgggcgatttcctcgcgtcgctcaaggatgcggacgggcggccggtctccttcatcctccgtccctggcacgagcacatcggcagctggttctggtggggcggccgtctctgctccgagcagcagtacaaggacctgttccgcctgacccacgactacctgaccgtcacccgcggtttgacggacatcgtctggtgctattccccgaactccggaatttcgccacagcagtacatgagccggtatccgggcgacgactgtgtcgatatcctcggaatggacgcttatcagtatgtgggcgatcagccgctggaggatgcggagaccgctttcgtcgcacaggtccgggaccacctggccttcatgaaagacctggcgcaggagcacgggaaactgatgtgcctgtccgagaccggattcgaaggcatccccgatcctgcctggtggaccggcaccctgctcccggccatccaggattttccgattgcgtatgtcctgacctggcgcaatgcccacgacaagccggaacatttctatgccgcctggcccggatcgggtgacgaagcggatttcaaggcttttgccgaaaaggataacatcctgttcctgaacgagtaa

SEQ ID NO: 3 - DNA, 1068 bp, Unknown
  Description of sequence sequence from genomic DNA library from rumen ecosystem, particularly nucleic acid sequence of clones pBKRR 2 and 16; RR02-1 (First enzyme) (= RR16); CDS; (see Figure 10)
atggctttgacctcatgcggtaccggaacaaaaaagcccatggaaacgcccaaagaccagctggttcaccagttgttcacctatgctgcgaaagggcagatcgcttacggccaccaggacgacctggcctatggccacaattgggtggtgaccgactgggagaacgacccgctggagcgctcggacgtgaaggccgttaccggcaagtaccccgccgtcgtggggttcgacctgggcggcatcgagctgggccacgccaagaatctggacggcgtgccgttcggcctgatgcgaaaggcggcgcagaagcacgtggagcgtggtggcattgtcaccttctcctggcatccgcgcaatccgcttaccggcggcgatgcctgggacatcagctccgaccaggtggtgaaatcggttctgaccggcggggagaagcatggcgtttttatgctttggctcactcgtgcggccgattttatcgaaagactgggggccgatgtccccgtgattttccgcccctggcacgagaacctgggaagctggttctggtggggaaagaacctctgcacggaacaggaataccaggagctttaccgcatgacctggctctatttcaccaaggagcgtggcctgaccaacatcctgtggtgctattcgcccaacggtccgatagaaccggaactgtatatgtcccgctatcccggcgatgaattcgtggatatcctggggacggatatttatgagtatgtcggggccgacggcctggaagaagccggcgtgcgttttagtatggaggtgaaaagtatgctgacggccatgaatgtaatggcgacggatcaccacaaactcatgtgcctgtcggaaaccggcctggaaggcatccccagccccacttggtggacgggcgtactggaacccgccatccgggaattccccatcagttatgtgctcacctggcgcaacgcccatgatatccctacgcatttctatgcggcctgggagggctttgaacacgctgccgacatgaaggcgttcagcgagttggacaatattgtatttttagacgaataa

SEQ ID NO: 4 - DNA, 1599 bp, Unknown
  Description of sequence sequence from genomic DNA library from rumen ecosystem, particularly nucleic acid sequence of clones pBKRR 2 and 16; RR02-2 (Second enzyme) (= RR16); CDS; (see Figure 10)
atgaaaacgatccgttccatagccttttgcaccgcggtgcttgccctgctttctacagcttgccagaagcctgatccaaccccgacccccgtagatgaagcccccacttccatcatgctcagccagagcagtttttctgtggcaaataccggagagaaactgtccttgacggtaacggcgccagccaggcctgccgtttccggcctgccggactggatttccttgacggatggaacctattccaagtacaagattacattcggtcttatggtagcggccaatgccggttatcaggagcggacggccaccctgaccgtcacctcttccggcgcttcttccgtgactgttaccgtgacgcaggccgcttccgcagaaccggagccgcccacctccgatccggacccggataataaccctgcatggaaaatggcgatgaacctgggccttggatggaacatgggcaaccagtttgacggctattacaacggctcctgggcgggagagaaagaaggatatccaggggagtgtgtctggcagcctgatgacagccataaggccacgcaggctactttcgaagggctgaagaaagccgggttcaccagcgtccgtattccggtgagctggcttcggatgattggtccggctcctgcctacaccatcgatgaaacctggcttggcagggtgtacgaagtggtcggattcgcacacaatgcagggcttaacgtgattgtgaatacgcatcatgacgagaaccatggagtaaacaatacgtaccagtggctggatatcaagaatgctgccaataattccgctctgaaccaggccatcaaggaggaaatcaaggcggtctggacccagattgccgaaaagttcaaagactgcggcgactggctcatcctggagagtttcaatgagttgaacgacggtggctggggctggagcgatgctttccgggccaatccgtccaagcagtgcgatatcctgaacgagtggaatcaggtcttcgtggatgccgtccgggccaccggcggagaaaatgccacgcgctggctgggcgttcccacttatgcggccaacccggaatacactacttatttcacgatgccctctgatcctgccggaaagacaatgctggccgtccatttctacgacccctccgactataccatcggcaaggagcagtactccgactggggacataccggccaggccgggcagaaagctacctggggcgatgaagaccatgtacgggaagtgttcggaaaactgaacgccagttttgtcgaaaagaagattccagtctatctgggcgaattcggctgctcgatgcgtcgcaaatccgacagccgtgcctgggctttctacaagtactatctggagtatgtggtaaaagcggccaggacctatggcctcccgtgtttcctgtgggacaacggcggcaccgactccggccaggaacagcatggatatatccatcatggtaacggcagttatctcggaaacagcaaggagctcgtcgacttgatggtgaaagcctggtttacagataaggaaggatataccctccagaccatctacaacagtgctcccaagttctga

SEQ ID NO: 5 - DNA, 1548 bp, Unknown
  Description of sequence sequence from genomic DNA library from rumen ecosystem, particularly nucleic acid sequence of clones pBKRR 22, 6, 8 and 23; (see Figure 11)
atgctgttggcatctttctcattgtttagttgtggcggaagcgatggagacgactccacgccgacacccagcgtaagcctgcagctagtttcgcaatcgatagccgaaggtgcccaggtggatgctgcatcgactactgtccttacactgaattataacaaccctgtcagagtgaccggaaatggtgttaccctcaatggaatagcagtgaaagccaaggtttcgggcaacacatctgtagaaatcccgcttacgctggtagaaggcacggtctatacgctgaaggtggcgtggggtgccatcgtggctgccgctgacggaaaagttgtggctcctgagtttacgctgaacttcactaccaagggaaatcagcaacctcccatcgacgagaacagtcctatcgccaagaaaatgggctggggatggaacctgggcaaccatttcgacaccagcagcggtgctgacggtgtgcgtccgcagtggggatattgggacaatgccaaacccacacaggtcctctacaacaaccttagaaacgctggaaccagcaccgtacgtatcggcgtaacatggggcaactaccagaatacatcgaactgggacatagatgctaactacatagcagaagtcaggcagaatgtggagtgggccgaagctgccggactgaacgttatcctgaacatgcaccacgatgagtactggctcgacattaagggcgctgccaacaattcgtcgaccaacaccaatatcaagaaccgtatcgagaaaacctggaaacagattgccgaagccttcaaggacaagggcgagtttctcatcttcgagtcgttcaacgagattcaggacggcggctggggatggggcgcaaaccgtaccgacggcggtaagcagtataaaacccttaacgaatggaaccagttggtggtgaacaccattcgtgccaccggcggcaacaacgccacccggtggataggcataccttcttacgccgctaatccttcgctcgcactcgaaaccggcttcgtgcttcctaccgatgctgccaaccgcctgatggtcagcgtccacttctacgaccccagcaacttcaccctgtcaccctatgttagcgatgacagcagtgagtataaaagtggtggctacagcgagtggggccacactgccgctaagggcaaggccgatactggcagcaacgaagaccatgtagtagctattttcaagaagttgcacgataagttcgtggctaagaatattcccgtttacatcggcgagtacggttgcgtgatgcacaagaacacccgttccaactacttccgcaactactatctcgagtatgtatgccgtgcagcccatgagtatgggatgccgctctgcatctgggataacaaccaaacgggtggcggcaacgagcatcatggctattttaaccacaccgacggtcagtatctcaatgccatggagtcgcttgtgaagacaatgatcaaggccgccaccagcgatgatgccagttacaccctcgagagcgtctataacaaagctccgaagtaa

SEQ ID NO: 6 - DNA, 1995 bp, Unknown
  Description of sequence sequence from genomic DNA library from rumen ecosystem, particularly nucleic acid sequence of clone pBKRR 7; RR07; CDS;(see Figure 12)
gtgatcgcagcggtcctattggtaggggccgttttgatagaaaagtacggtaacctggctacatggcaacttttgctggtttatcttgtcccctacttgtacatcggatacgacacactgaaagaggcagcagaaggtatcgcccacggtgatgctttcaacgagcacttcttgatgtcgattgccaccctcggagctttagccatcggttttctgccaggtgcagagacacagtttcccgaggccgtcttcgtgatgctcttcttccaggtgggtgagctgtttgagggctacgccgaagggaagagtcgcgacagcattgcccatctgatggatatccgtccggatgtggctcaccttctcgacgaaggaggaaggacgaaggaggaaggagagtccacccctaacccctcccaaagggagggagatgtgaaggatgtgcgtcctgagaatgtggaggtgggttctatcatcctgatccgccctggtgagaagattccactagacggtgtggttctggagggtagttcagctttgaacacggtggcgctgactggtgagagcatgccacgcgacattactgttggcgatgaggtgatctcaggatgcatcaacctgtcgggggttatcaaggtgcgtaccaccaagagttttggtgagagtaccgtatcgaagatcatcagtctggtagaacatgccagcgagcacaagtcgaagagcgaagcattcatcagtaagttcgcccgtatctacaccccgatagtggtctttgccgccctcgccctggccattctccccccactattggctgccatcaccatttccccatttaattttcaattgtcaattttcaattctcaattttctacctggctctaccgtgccctgatgttcttggtggtgtcttgtccttgcgcactggtcatcagcgttcccctcacctttttcggcggtatcggcggtgcgtcacgcaaaggtatcctcatcaagggcgccaactacatggatatcctggccaaggtgaagaccgttgtctttgataagaccggtaccctcacccacggacaatttgccgtagaagctgtgcatcctaatcaaggggagtcatcatgtagtaattctgcagcagcagactctcacctctcatctctcatctctcatctccttcatctggccgcccatgtggaaagtttctcctcccaccccattgctgccgccctgcgtgatgcttttcctgaggcagctcacgacgattgcgaggtgagcgatatcaaggaaatcgctggtcatggcattcaggcccgggtgggcaatgacatcgtttgtgtgggtaacacgaagatgatggacagcttgggtgttgcctggcacgactgtcatcatcctggaaccatcatccatgtggccatcaacggggaatatgccggacatatcatcatcaacgacatgatcaaggacgacagtgccgaagccatccgacaactgaaggagttgggagtggaaagaaccgttatgcttacgggcgaccgtaaagaggtagccgaccatgtggctaagatgttggatatcactgaataccatgccgaactcctccccacagacaaagtttctttcgttgagcaagagctgtctgctcactcttcttccacgtctcacccctcatatgtggcctttgtcggcgacggtatcaatgatgctcctgtactggctcgagctgatgtgggtatcgccatgggcggattgggtagtgatgctgccattgaagcagccgacgtggttctgatggacgatcagccctcgaagattgcccaggctatccgcattgcccgtcgcactctttccatcgcccgacagaacgtgtggttcgccatcggtgtaaaaatcgccgtactcatcctcgccaccttcggcctcagcaccatgtggatggccgtctttgccgatgtgggcgtcaccgtactggccgtactcaacgccatgcgaaccatgtattaa

SEQ ID NO: 7 - DNA, 1062 bp, Unknown
  Description of sequence sequence from genomic DNA library from rumen ecosystem, particularly nucleic acid sequence of clone; pBKRR 9; RR09; CDS;(see Figure 13)
atgaaattagtagagtttaacgcagaagaaaactatatagaagattttttaagtttacctaaaaaattatatacaaaattagataatatggaagattctaacactatgaaagatattcttactaataatcatcctttaagtaacgattttaaattaaataaatatttagtatataaagatgatgaagtagtcgcaaggtttataataactgaatacaatgatgataataaagtgtgttatataggattttttgaatgtattaatgataaaaaagtagctaagttcttatttgatgaagctcacaaaatagctaaagaaaagaaatataaaaagattattggtccagtagatgcatccttttggattaaataccgtctaaaaataaataaatttgaaagaccctataccggagaaccatataataaagattattatttaaagttatttacagataataaatataaaatatgtgatcattatacttcacaaagctatgatatagtagatgatagttattttaatgataaatttactgaacactataatgaatttaaaaaactaggttatgaaataattaaacctaaaccagaagattttgataaatgtatgtcagaagtatatgatttaattactaagttatatagcgattttcctatatttaaaaatttaaaaaaagaatctttcttagaaatatataaatcttataaaaaaattattaatatgaatatggttagaatggcatattttaaaggacaagcagtaggtttctatataagtgtacctaactataataatattgtatatcatattaatcccataaatatattaaagattcttcatcaaagaaaacacccaaaaggctatgtaatgctttatatgggagtagattcaaatcatagaggattaggtaaagcattagtatattcaatagtagaagaattaaagaaaaataatctaccaagtattggagctttagctcatgatggtaaaattagtcagaattatgcaaaagaaaaaataaattctagatatgaatatgtattacttgaaaggagtatttaa

SEQ ID NO: 8 - DNA, 1611 bp, Unknown
  Description of sequence sequence from genomic DNA library from rumen ecosystem, particularly nucleic acid sequence of clones pBKRR 11, 10 and 12; RR10=RR11=RR12; CDS;(see Figure 14)
atgagaattttaaaattattttttattgcgcttataacagctgctccaatcttcagttatgctcagacagtcgatgatatgaaatcagtctttgttgatgcccacacgtggacagcctctataaaaatgggttggaatttgggtaatgcccttgaatgtcagacaggatggaatgataaggcgttttgttggaatcctgcaaacaactttaatgcagagacctcgtggggaaatcccaaaaccacaaaagaaatgcttcaggctgttcgtgaggcaggttttgatgctgtgcgtattcctgtaaactggggtgcccatataagcgatgagtcaacttgtactattgatgcagcttggatgaatcgtgttcaagaagttgttgactgggctctgcaacttggtttcaaggtattgctcaatacccatcatgagtattggcttgagtggcatccaacctatgaccgtgaacaggctaataataataaacttgctaagatatggatgcaggttgctaatagatttaaggattatggtccagaccttgcatttgccggtatcaacgaggttcatttggatggtaagtggaatgttccaacagaagagaatactgcagttaccaatagttataatcagacatttgttaataccgttcgtgcaacaggtggcaataacttatatcgtaatcttgtagtacaatgttattcatgtaatccagattacggcttgacaggttttgttgttcctgctgacaaggttgagaatcgtcttagcatagaatatcattattatcgtccatgggattatgccggcgatgctccaaagtattactattggggtgaggcgtataagcaatatggcgaggtgagcacagccgacaacgaggcaaaggttaatgccgattttgcacgttacaaggcagcttggtatgacaagggcttgggtgtgattatgggtgaatggggtgcgagtcagcattatcaggtagagagcaaggcaaaacagcttgaaaacctatattaccatttcaagacagttcgtgaggcagcacagaaaaataatattgctacttttgtatgggataataatgcatgtggaaatggtggtgacaagtttggaatttttgatagaaatgacaatatgtctgtagcgttcccagaagcactaaatgctattcaggaggcatcgggacaaacggttgttgactatagcagacaggctttgcagccttctgtggtttatcagggtgatgagctaatgaattggggtgaaggcaagcagttgattattgctggaagtaagtttgcttattttaccgcagagagcaaacttatggttactcttgatgctgaaccaggtgcagattatgatatgttgcagtttgcttatggcgactggaaatcaaagccattgatgattatttcgggcaggagttacaagggacaggttgagccttcaaagattaatggttctcgcaatacatataccctgtttattggttttaaggaatcgtctcttaaccaactgaaggataagggaataacccttcaggggcatggtcttcgtttgcgtaatgtagttgtgatgccagaagggcttgtattagggttataa

SEQ ID NO: 9 - DNA, 963 bp, Unknown
  Description of sequence sequence from genomic DNA library from rumen ecosystem, particularly nucleic acid sequence of clone pBKRR 13; RR 13; CDS;(see Figure 15)
atgacaatgaagaaactactgaccatcattatgttgagtttgttggcatgtcacgtacacgccaacgaccccgtcaagcagtacggaaaactgcaagtaaaaggtgcccagctctgcgaccaaaagggcaatcctgtcattctgcgtggtgtcagtctgggctggcacaacctgtggccgcgcttctataacaagggggccgtggagacgctgaagaacgactggcactgtagtgtcatccgtgcggctatcggacagcatatcgaggacaacttccaggagaaccccgagttcgccatgcagtgtctgacgcccgtcgttgaagccgccatcaagcagaacgtctacgtcatcatcgactggcactcgcacaaactgatgacggaagaggccaagcagttctttggcgagatggcccggaaatacggcaagtatccgcacatcatctacgaaatctacaacgagcccatcgcggacacgtggaccgatctgaagaaatacgctcaggaggttattggagaaatccgcaaatacgataaggacaatgtcgtactcgtgggttgcccccactgggatcaggatatccatctggttgccgagagtccgttggagggcctctcgaacgtcatgtacaccgtgcatttctatgccgctacgcatggtgaagacctgcgtcagcgtacagaagaagctgccaaacgaggtattcccatcttcatttccgaaagcggtgccaccgaggccagcggcgacggtaagattgatgaggagagcgaggaggaatggatcaagatgtgcgaacgcctgggcatcagctggctgtgctggagcatcagcgacaagaacgagtcgagttcgatgctgctgcctcgtgccactgctacaggtccttggcccgatgacgtcatcaagaaatacggcaaactggtgaagggactgctggagaaatataatgcgaggtaa

SEQ ID NO: 10 - DNA, 1452 bp, Unknown
  Description of sequence sequence from genomic DNA library from rumen ecosystem, particularly nucleic acid sequence of clones pBKRR 14 and 20-1; RR14=RR20-1; CDS;(see Figure 16)
atgatcaatgtcctgcagcatcatcccgaggtggagctgtcggtggacaaaacttccgtctccttcaaccgttccgggggagaggagaccttcacggtcacttcttccacccagcctcatgtgtcggccgacgtttcctgggttgtggtcgagacgggcaagattgacaaggatcaccataccgaagtcagggtcttggcgggggcgaaccggaaggaggcttccgcaggaaccttgacggtgtcctgcagcgacaagaaagtgtcggtcagcgtcaagcaggaggcatttgtcgctccttccgtcgccagcacgacggcggtgactccgcagatggtgttcgatgccatgggaccgggctggaacatgggcaaccacatggacgccatcagcaacggcgtgtccggcgagacggtctggggcaatcccaaatgcacccaggccacgatggacggggtcaaagcggccggttacaaggccgtccgcatctgcacgacctgggaaggccatatcggcgccgctccggcgtatgcgctcgagcagaaatggctcgaccgcgtggcggagattgtcgggtatgccgaaaaagccggcctcgtcgccatcgtgaacacgcatcatgacgagagctattggcaggacatcagcaagtgctataacaacgcggcgaatcacgagaaggtcaaggacgaggtgttcagcgtctggacccagatcgcggagaagttcaaggacaagggcgaatggctcgtgttcgagtccttcaacgagatccaggacggcggctggggctggagcgacgccttccggaagaatccggacgcgcagtacaaggtgctcaatgaatggaaccagaccttcgtggacgcggtccgcagcacgggcgggcagaatgcgacccgctggctcggaattccgggctacgcctgcaacccgggcttcacgatcgccggtctggtgctgcccaaggattacactaccgccaaccgcctgatggtggccgtccatgactacgacccgtacgactatacgctcaaggatccgctcatccgccagtgggggcatacggccgatgcggacaagcgcccgagcggggacaacgagaaggctgtcgtggatgtcttcaacaatctcaaggccgcctacctggacaagggtattccggtctatctcggcgagatgggctgctcacgccataccgccgcggacttcccgtatcagaagtattacatggagtatttctgcaaggccgccgccgaccgcctgctgccgatgtacctctgggacaacggcgccaagggtgtcggctcggagcgccacgcctacatcgaccacggaaccggacagttcgtggacgaggatgcccggacgcttgtggggctgatggtgaaggccgtgacgacgaaagatgcgtcctacacgctggagagcgtctataactccgcgccgtaa

SEQ ID NO: 11 - protein, 528 aa, Unknown
  Description of sequence sequence derived from genomic DNA library from rumen ecosystem, particularly amino acid sequence of clones pBKRR 1 and 21; RR01-1 First enzyme (= RR21) ; (see Figure 17)
MKLYWKYLSLTAVLAVLASCGGNADPDPEPTPSAPSSITLSVNSFTVAQAGETLSLDITAPTRPKLALPNWITLKDGTYQNYKITVGLTVSANDTYQAREATVTVSATGTFDVTFKVSQAGKEPEPTPPDPSGGDNDAWQMAKKLGLGWNMGNHFDAFYNGSWAGEKEGYPDEEVWGASKATQTTFNGVKNAGFTSVRIPVSWLKMIGPAPEYKIDETWLNRVYEVAQYAHNAGLTAIVNTHHDENHGVDNTYQWLDIKNASTDASVNTRIKEEIKAVWTQIANKFKDCGDWLILESFNELNDGGWGWSDAFRANPTKQCNILNEWNQVFVDAVRATGGNNATRWLGVPTYAANPEFEKYATLPTDSANKIMLAVHFYDPSDYTIGDAQYSDWGHTGAADKKASGGDEDHVRSVFGNLCTKYVENNIPVYLGEFGCSMRAKSNTRAWKFYLYYMEYIVKAAKTYGLPCYLWDNGSKSEPGKEVHGYIDHGTGDYIGNSKEVIDVMKKAWFTESAGYTLQTVYNAAPKL

SEQ ID NO: 12 - protein, 383 aa, Unknown
  Description of sequence sequence derived from genomic DNA library from rumen ecosystem, particularly amino acid sequence of clones pBKRR 1 and 21; RR01-2 Second enzyme (= RR21) ; (see Figure 17)
MLRRNCNHLNMKHIALYFMLGAVALAAPGCGRRAERPATTPKDQVIAQLQEAVNGGKILYAHQDDLVYGHTWKVEDVAGDPLERSDVKAVTGFYPAMVGFDLGGIEMGWEANLDGVPFDLMRRAAVTHASRGGIVTFSWHPRNPLIGGDAWDVSSDQVVASILEGGEKHDLFMEWLRRAGDFLASLKDADGRPVSFILRPWHEHIGSWFWWGGRLCSEQQYKDLFRLTHDYLTVTRGLTDIVWCYSPNSGISPQQYMSRYPGDDCVDILGMDAYQYVGDQPLEDAETAFVAQVRDHLAFMKDLAQEHGKLMCLSETGFEGIPDPAWWTGTLLPAIQDFPIAYVLTWRNAHDKPEHFYAAWPGSGDEADFKAFAEKDNILFLNE

SEQ ID NO: 13 - protein, 332 aa, Unknown
  Description of sequence sequence derived from genomic DNA library from rumen ecosystem, particularly amino acid sequence of clones pBKRR 2 and 16; RR02-1 (First enzyme) (= RR16) ;(see Figure 18)
MALTSCGTGTKKPMETPKDQLVHQLFTYAAKGQIAYGHQDDLAYGHNWVVTDWENDPLERSDVKAVTGKYPAVVGFDLGGIELGHAKNLDGVPFGLMRKAAQKHVERGGIVTFSWHPRNPLTGGDAWDISSDQVVKSVLTGGEKHGVFMLWLTRAADFIERLGADVPVIFRPWHENLGSWFWWGKNLCTEQEYQELYRMTWLYFTKERGLTNILWCYSPNGPIEPELYMSRYPGDEFVDILGTDIYEYVGADGLEEAGVRFSMEVKSMLTAMNVMATDHHKLMCLSETGLEGIPSPTWWTGVLEPAIREFPISYVLTWRNAHDIPTHFYAAW

SEQ ID NO: 14 - protein, 532 aa, Unknown
  Description of sequence sequence derived from genomic DNA library from rumen ecosystem, particularly amino acid sequence of clones pBKRR 2 and 16; RR02-2 (Second enzyme) (= RR16) ;(see Figure 18)
MKTIRSIAFCTAVLALLSTACQKPDPTPTPVDEAPTSIMLSQSSFSVANTGEKLSLTVTAPARPAVSGLPDWISLTDGTYSKYKITFGLMVAANAGYQERTATLTVTSSGASSVTVTVTQAASAEPEPPTSDPDPDNNPAWKMAMNLGLGWNMGNQFDGYYNGSWAGEKEGYPGECVWQPDDSHKATQATFEGLKKAGFTSVRIPVSWLRMIGPAPAYTIDETWLGRVYEVVGFAHNAGLNVIVNTHHDENHGVNNTYQWLDIKNAANNSALNQAIKEEIKAVWTQIAEKFKDCGDWLILESFNELNDGGWGWSDAFRANPSKQCDILNEWNQVFVDAVRATGGENATRWLGVPTYAANPEYTTYFTMPSDPAGKTMLAVHFYDPSDYTIGKEQYSDWGHTGQAGQKATWGDEDHVREVFGKLNASFVEKKIPVYLGEFGCSMRRKSDSRAWAFYKYYLEYVVKAARTYGLPCFLWDNGGTDSGQEQHGYIHHGNGSYLGNSKELVDLMVKAWFTDKEGYTLQTIYNSAPKF

SEQ ID NO: 15 - protein, 515 aa, Unknown
  Description of sequence sequence derived from genomic DNA library from rumen ecosystem, particularly amino acid sequence of clones pBKRR 22, 6, 8 and 23;(see Figure 19)
MLLASFSLFSCGGSDGDDSTPTPSVSLQLVSQSIAEGAQVDAASTTVLTLNYNNPVRVTGNGVTLNGIAVKAKVSGNTSVEIPLTLVEGTVYTLKVAWGAIVAAADGKVVAPEFTLNFTTKGNQQPPIDENSPIAKKMGWGWNLGNHFDTSSGADGVRPQWGYWDNAKPTQVLYNNLRNAGTSTVRIGVTWGNYQNTSNWDIDANYIAEVRQNVEWAEAAGLNVILNMHHDEYWLDIKGAANNSSTNTNIKNRIEKTWKQIAEAFKDKGEFLIFESFNEIQDGGWGWGANRTDGGKQYKTLNEWNQLVVNTIRATGGNNATRWIGIPSYAANPSLALETGFVLPTDAANRLMVSVHFYDPSNFTLSPYVSDDSSEYKSGGYSEWGHTAAKGKADTGSNEDHVVAIFKKLHDKFVAKNIPVYIGEYGCVMHKNTRSNYFRNYYLEYVCRAAHEYGMPLCIWDNNQTGGGNEHHGYFNHTDGQYLNAMESLVKTMIKAATSDDASYTLESVYNKAPK

SEQ ID NO: 16 - protein, 664 aa, Unknown
  Description of sequence sequence derived from genomic DNA library from rumen ecosystem, particularly amino acid sequence of the polypeptide and the mature protein of clone pBKRR 7; Polypeptide ;(see Figure 20)
VIAAVLLVGAVLIEKYGNLATWQLLLVYLVPYLYIGYDTLKEAAEGIAHGDAFNEHFLMSIATLGALAIGFLPGAETQFPEAVFVMLFFQVGELFEGYAEGKSRDSIAHLMDIRPDVAHLLDEGGRTKEEGESTPNPSQREGDVKDVRPENVEVGSIILIRPGEKIPLDGVVLEGSSALNTVALTGESMPRDITVGDEVISGCINLSGVIKVRTTKSFGESTVSKIISLVEHASEHKSKSEAFISKFARIYTPIVVFAALALAILPPLLAAITISPFNFQLSIFNSQFSTWLYRALMFLVVSCPCALVISVPLTFFGGIGGASRKGILIKGANYMDILAKVKTVVFDKTGTLTHGQFAVEAVHPNQGESSCSNSAAADSHLSSLISHLLHLAAHVESFSSHPIAAALRDAFPEAAHDDCEVSDIKEIAGHGIQARVGNDIVCVGNTKMMDSLGVAWHDCHHPGTIIHVAINGEYAGHIIINDMIKDDSAEAIRQLKELGVERTVMLTGDRKEVADHVAKMLDITEYHAELLPTDKVSFVEQELSAHSSSTSHPSYVAFVGDGINDAPVLARADVGIAMGGLGSDAAIEAADVVLMDDQPSKIAQAIRIARRTLSIARQNVWFAIGVKIAVLILATFGLSTMWMAVFADVGVTVLAVLNAMRTMY

SEQ ID NO: 17 - protein, 324 aa, Unknown
  Description of sequence sequence derived from genomic DNA library from rumen ecosystem, particularly amino acid sequence of the polypeptide and the mature protein of clone pBKRR 7 mature protein (according to the protein homology analysis) ; (see Figure 20)
VKTVVFDKTGTLTHGQFAVEAVHPNQGESSCSNSAAADSHLSSLISHLLHLAAHVESFSSHPIAAALRDAFPEAAHDDCEVSDIKEIAGHGIQARVGNDIVCVGNTKMMDSLGVAWHDCHHPGTIIHVAINGEYAGHIIINDMIKDDSAEAIRQLKELGVERTVMLTGDRKEVADHVAKMLDITEYHAELLPTDKVSFVEQELSAHSSSTSHPSYVAFVGDGINDAPVLARADVGIAMGGLGSDAAIEAADVVLMDDQPSKIAQAIRIARRTLSIARQNVWFAIGVKIAVLILATFGLSTMWMAVFADVGVTVLAVLNAMRTMY

SEQ ID NO: 18 - protein, 353 aa, Unknown
  Description of sequence sequence derived from genomic DNA library from rumen ecosystem, particularly amino acid sequence of clonepBKRR 9; RR09;(see Figure 21)
MKLVEFNAEENYIEDFLSLPKKLYTKLDNMEDSNTMKDILTNNHPLSNDFKLNKYLVYKDDEVVARFIITEYNDDNKVCYIGFFECINDKKVAKFLFDEAHKIAKEKKYKKIIGPVDASFWIKYRLKINKFERPYTGEPYNKDYYLKLFTDNKYKICDHYTSQSYDIVDDSYFNDKFTEHYNEFKKLGYEIIKPKPEDFDKCMSEVYDLITKLYSDFPIFKNLKKESFLEIYKSYKKIINMNMVRMAYFKGQAVGFYISVPNYNNIVYHINPINILKILHQRKHPKGYVMLYMGVDSNHRGLGKALVYSIVEELKKNNLPSIGALAHDGKISQNYAKEKINSRYEYVLLERSI

SEQ ID NO: 19 - protein, 536 aa, Unknown
  Description of sequence sequence derived from genomic DNA library from rumen ecosystem, particularly amino acid sequence of clones pBKRR 11, 10 and 12; RR10=RR11=RR12; (see Figure 22)
MRILKLFFIALITAAPIFSYAQTVDDMKSVFVDAHTWTASIKMGWNLGNALECQTGWNDKAFCWNPANNFNAETSWGNPKTTKEMLQAVREAGFDAVRIPVNWGAHISDESTCTIDAAWMNRVQEVVDWALQLGFKVLLNTHHEYWLEWHPTYDREQANNNKLAKIWMQVANRFKDYGPDLAFAGINEVHLDGKWNVPTEENTAVTNSYNQTFVNTVRATGGNNLYRNLVVQCYSCNPDYGLTGFVVPADKVENRLSIEYHYYRPWDYAGDAPKYYYWGEAYKQYGEVSTADNEAKVNADFARYKAAWYDKGLGVIMGEWGASQHYQVESKAKQLENLYYHFKTVREAAQKNNIATFVWDNNACGNGGDKFGIFDRNDNMSVAFPEALNAIQEASGQTVVDYSRQALQPSVVYQGDELMNWGEGKQLIIAGSKFAYFTAESKLMVTLDAEPGADYDMLQFAYGDWKSKPLMIISGRSYKGQVEPSKINGSRNTYTLFIGFKESSLNQLKDKGITLQGHGLRLRNVVVMPEGLVLGL

SEQ ID NO: 20 - protein, 320 aa, Unknown
  Description of sequence sequence derived from genomic DNA library from rumen ecosystem, particularly amino acid sequence of clonepBKRR 13; RR 13;(see Figure 23)
MTMKKLLTIIMLSLLACHVHANDPVKQYGKLQVKGAQLCDQKGNPVILRGVSLGWHNLWPRFYNKGAVETLKNDWHCSVIRAAIGQHIEDNFQENPEFAMQCLTPVVEAAIKQNVYVIIDWHSHKLMTEEAKQFFGEMARKYGKYPHIIYEIYNEPIADTWTDLKKYAQEVIGEIRKYDKDNVVLVGCPHWDQDIHLVAESPLEGLSNVMYTVHFYAATHGEDLRQRTEEAAKRGIPIFISESGATEASGDGKIDEESEEEWIKMCERLGISWLCWSISDKNESSSMLLPRATATGPWPDDVIKKYGKLVKGLLEKYNAR

SEQ ID NO: 21 - protein, 483 aa, Unknown
  Description of sequence sequence derived from genomic DNA library from rumen ecosystem, particularly amino acid sequence of clones pBKRR 14 and 20-1; RR14=RR20-1;(see Figure 24)
MINVLQHHPEVELSVDKTSVSFNRSGGEETFTVTSSTQPHVSADVSWVVVETGKIDKDHHTEVRVLAGANRKEASAGTLTVSCSDKKVSVSVKQEAFVAPSVASTTAVTPQMVFDAMGPGWNMGNHMDAISNGVSGETVWGNPKCTQATMDGVKAAGYKAVRICTTWEGHIGAAPAYALEQKWLDRVAEIVGYAEKAGLVAIVNTHHDESYWQDISKCYNNAANHEKVKDEVFSVWTQIAEKFKDKGEWLVFESFNEIQDGGWGWSDAFRKNPDAQYKVLNEWNQTFVDAVRSTGGQNATRWLGIPGYACNPGFTIAGLVLPKDYTTANRLMVAVHDYDPYDYTLKDPLIRQWGHTADADKRPSGDNEKAVVDVFNNLKAAYLDKGIPVYLGEMGCSRHTAADFPYQKYYMEYFCKAAADRLLPMYLWDNGAKGVGSERHAYIDHGTGQFVDEDARTLVGLMVKAVTTKDASYTLESVYNSAP

(19) United States
(12) Patent Application Publication
(10) Pub. No.: US 2008/0261267 A1
(43) Pub. Date: Oct. 23, 2008

(54) Cellulases from Rumen

(76) Inventors: Manuel Ferrer, Madrid (ES); Peter Golyshin, Wolfenbuttel (DE); Olga Golyshina, Wolfenbuttel (DE); Tatyana Chernikova, Braunschweig (DE); Carsten Strompl, Evessen (DE); Kenneth Timmis, Wolfenbuttel (DE); Kieran Elborough, Franklin (NZ); Graeme Jarvis, Wellington (NZ)

Correspondence Address: KLAUBER & JACKSON, 411 HACKENSACK AVENUE, HACKENSACK, NJ, 07601, US

(21) Appl. No.: 11/631,243

(22) PCT Filed: Jun. 30, 2005
(86) PCT No.: PCT/EP2005/053104

§ 371 Date: Mar. 19, 2008

(30) Foreign Application Priority Data
Jul. 2, 2004 (EP) 04015680.4

(51) Int. Cl. C12P 21/04; C12N 9/42; C12N 15/11; C12N 15/00; C12N 1/20

(52) U.S. Cl. 435/69.1; 435/209; 536/23.2; 435/320.1; 435/252.33; 435/263; 435/267

(57) ABSTRACT

The invention provides polypeptides coding for new cellulases from rumen, particularly from rumen ecosystem. The invention also relates to functional fragments or functional derivatives thereof as well as to nucleic acids encoding the polypeptides of the invention, vectors and host cells containing said nucleic acids, a method for producing the polypeptides and the use of the polypeptides according to the present invention for various industrial purposes and medical treatments.

The invention relates to new cellulases from rumen, in particular to polypeptides comprising or consisting of an amino acid sequence according to FIG. 17 to 24 of the invention or parts thereof or a functional fragment or functional derivative thereof.

Cellulases or cellulolytic enzymes are enzymes which are involved in hydrolysis of cellulose, especially in hydrolysis of the 3-D-glucosidic linkages in cellulose. Cellulose is an unbranched polymer of 1,4-linked anhydrous glucose units of variable length, and is one of the most abundant natural polymers with an estimated annual production of 4×10⁹t. Cellulose offers an energy and carbon source for some organism, particularly microorganism. The cellulose polymer itself cannot pass the membrane of microorganisms and has to be hydrolyzed prior to utilization. Therefore, cellulases are synthesized by a large number of microorganisms which include fungi, actinomycetes, mycobacteria and true bacteria but also by plants.

In the hydrolysis of native cellulose, it is known that there are three major types of cellulase enzymes involved which form a cellulolytic system and hydrolyze insoluble cellulose, cellulose derivatives (such as carboxymethyl cellulose and hydroxyethylcellulose), lichenin, beta-1,4 bonds in mixed beta-1,3 glucans such as cereal beta-D-glucans or xyloglucans and other plant material containing cellulosic parts in a very efficient and synergistic way. These three types of cellulose enzymes are:
(a) endo-beta-1,4-glucanases (endo-1,4-beta-D-glucan-4-glucanohydrolase, EC 3.2.1.4) which randomly hydrolyze the 1,4-glycosidyl linkages within the water-insoluble cellulose chains,
(b) exoglucanases or cellobiohydrolases (1,4-beta-D-glucan-cellobiohydrolase, EC 3.2.1.91) which hydrolyze the 1,4-glycosidyl linkages at either the reducing or non-reducing ends of cellulose chains to form cellobiose (glucose dimer) and
(c) beta-glucosidases or cellobioses (EC 3.2.1.21) which convert the water soluble cellobiose into two glucose residues.

Especially type (a) the endo-beta-1,4-glucanases constitute an interesting group of hydrolases for industrial uses and thus a wide variety of specificities have been identified (T-M. Enveri, Microbial Cellulases in W. M. Fogarty, Microbial Enzymes and Biotechnology, Applied Sciences Publishers, p. 183-224 (1983); Methods in Enzymology, (1988) Vol. 160, p. 200-391 (edited by Wood, W. A. and Kellogg, S. T.); Béguin, P., Molecular Biology of Cellulose Degradation, Annu. Rev. Microbiol. (1990), Vol. 44, pp. 219-248; Béguin, P. and Aubert, J-P., The biological degradation of cellulose, FEMS, Microbiology Reviews 13 (1994) p. 25-58; Henrissat, B., Cellulases and their interaction with cellulose, Cellulose (1994), Vol. 1, pp. 169-196). Industrially well-performing endo-beta-1,4-glucanases are described in e.g. WO 91/17243, WO 91/17244 and WO 91/10732 and specific cellulase variants are described in WO 94/07998.

Cellulases or cellulolytic enzymes are mainly used for industrial purposes. Such industrial uses include, for example, the use in consumer products and food industries, e.g. in extracting and clarifying juice from fruits or vegetables. Another important industrial use of cellulases or cellulolytic enzymes is their use for treatment of paper pulp, e.g. for improving the drainage or for deinking of recycled paper. Even further cellulase applications include biodiesel production, insofar cellulases are needed to convert cellulosic biomass to fermentable sugars which are a valuable source for the production of chemicals and fuels, syrup production and agrobiotechnological processes, such as bioprocessing of crops and crop residues, fibre processing and production of feed supplements to improve feed efficiency.

However, among many applications which have been developed for the use of cellulolytic enzymes a very important industrial use of cellulases or cellulolytic enzymes is the use for treatment of cellulosic textile or fabrics, e.g. as ingredients in detergent compositions or fabric softener compositions for biopolishing of new fabrics (garment finish) or for obtaining a stone-washed look of cellulose containing fabrics, especially denim. Therefore, cellulases are known to be useful in detergent compositions for removing dirt, i.e., cleaning. For example, GB Application Nos. 2,075,028, 2,095,275 and 2,094,826 illustrate improved cleaning performance when detergents incorporate cellulases. Additionally, GB Application No. 1,358,599 teaches the use of cellulases in detergents to reduce the harshness of cotton containing fabrics. Another useful feature of cellulases in the treatment of textiles is their ability to recondition used fabrics by making their colors more vibrant. For example, repeated washing of cotton containing fabrics results in a greyish cast to the fabrics which is believed to be due to disrupted and disordered fibrils caused by mechanical action. This greyish cast is particularly noticeable on colored fabrics. As a consequence, the ability of cellulase to remove the disordered top layer of the fiber and thus improve the overall appearance of the fabrics has been of value.

Since detergents containing cellulases operate generally under alkaline conditions, there is a strong demand for cellulases which have excellent activity at pH 7-10. Well characterized fungal cellulases, such as those from Humicola insolens and Trichoderma reesei, perform adequately at neutral to low alkaline pH. Further, a number of bacterial enzymes that show cellulase activity at high alkaline pH have been isolated from Bacillus and other prokaryotes, see e.g., WO 96/34092 and WO 96/34108. Furthermore, Wilson et al., Critical Reviews in Biotechnology, Vol. 12, pp. 45-63 (1992), studied cellulases produced by Thermomonospora fussa, Thermomonospora curvata and Microbispora bispora and discovered that many of these cellulases show broad pH profiles and good temperature stability. Similarly, Nakai et al., Agric. Biol. Chem., Vol. 51, pp. 3061-3065 (1987) and Nakai et al., Gene, Vol. 65, pp. 229-238 (1988) exemplify the alkalitolerant cellulase casA from Streptomyces strain KSM-9 which also possesses an alkaline pH optimum and good temperature stability.

The enzymatic properties of some of the investigated cellulases in the art may fulfill the essential requirements upon which various industrial processes are based. However, apart from a high alkaline pH and a good temperature stability other properties are desirable for an effective application. Other properties are e.g., optimal phenotype of expression, temperature stability and pH optimum, high stability and high specific activity towards cellulose substrates, e.g. detergents, high enzyme rate and low addition of cations, because removal of these compounds is an expensive step of the overall process costs.

Despite vast information available in the art about numerous cellulases having desirable properties for certain applications, cellulases, cellulase compositions or cellulolytic enzymes which simultaneously exhibit some or even most of the aforementioned properties are not known.

Therefore, it is an object of the present invention to provide cellulases with improved properties, preferably a combination of improved properties, which are useful for industrial applications.

As a solution to the aforementioned object new cellulases which possess improved properties and which are advantageous for known applications of cellulose have been discovered. Therefore, the present invention relates in its first embodiment to a polypeptide comprising one of the amino acid sequences of amino acids No. 175 to No. 210 of the sequences shown in FIGS. 17 to 24 or a functional fragment, or functional derivative thereof.

Preferably, the polypeptide of the invention comprises one of the amino acid sequences of amino acids No. 175 to 210, preferably No. 170 to No. 220, more preferably No. 150 to 240, most preferably No. 120 to 280 of the sequences shown in FIGS. 17 to 24. More preferably, the polypeptide of the invention comprises one of the amino acid sequences shown in FIGS. 17 to 24.

The invention is based on the discovery that rumen ecosystems represent a unique microbial ecosystem with a high potential of microbial and manifold enzymatic diversity including, e.g., cellulases, hemicelluloses, xylases, glucosidases, endoglucanases etc. Therefore, rumen ecosystems containing a wide variety of microorganisms form a good starting material for screening new cellulases to obtain new cellulolytic activities. Consequently, according to a preferred embodiment of the invention, the polypeptide is derived from rumen, particularly from rumen ecosystem, preferably from cow rumen, more preferably from New Zealand dairy cow.

According to the invention it was possible to identify a group of endo-beta-1,4-glucanases from an expression library created previously from extracted rumial ecosystem-DNA. Especially, five novel high performance endo-beta-1,4-glucanases which hydrolyze i.a. carboxymethyl cellulose (CMC) were defined. Most of the polypeptides of the invention show an endoglucanase activity which is considerably higher than of endoglucanases known in the art (see i.a. FIG. 2, 3, 5, 6), are stable over a broad pH rang from pH 3.5 to pH 10.0 and at a temperature of up to 70° C. and are not influenced by mono- and divalent cations (see FIG. 3).

Consequently, in preferred embodiments of the invention functional active polypeptides show activity at pH optimum, preferably at pH ranging from 3.5 to 10.0, more preferably from 3.5 to 5.5 or from 5.5 to 7.0 or from 6.5 to 9.0, most preferably from 7.0 to 10.0, at temperature optimum, preferably at a temperature from 40° C. to 70° C., more preferably from 40° C. to 60° C., most preferably from 50° C. to 70° C. and/or at low addition of cations, preferably without any addition of cations. Furthermore, it is preferred that the polypeptides show highly specific activities and/or high stability towards its substrate. Some or all of these features may be realized by functional active polypeptides of the invention. In a particularly preferred embodiment of the invention, the polypeptide shows a combination of at least two, preferably three, more preferably four, most preferably all five of the aforementioned features.

Activity, i.e. hydrolyzing activity of the polypeptide of the invention, at a “pH optimum” means that the polypeptide shows activity and is stable towards its substrate at a pH which is optimal for the individual application. For use at acidophil conditions it is desired to obtain stable and specific activity from about pH 3.5 to 4.0, whereas use at alkaline conditions (e.g., in detergent compositions) needs a pH optimum from 9.0 to 10.0.

Correspondingly, activity at a “temperature optimum” depends on the specific use of the functional active polypeptides of the invention and means that the polypeptides show activity and are stable towards their environment conditions at a temperature which is optimal for the respective application. Usually, a temperature stability is required from 40° C. up to 70° C. for the functional active polypeptides of the invention. For most industrial applications a temperature stability of the functional active polypeptide according to the invention at 70° C. is preferred.

Most enzymes, particularly hydrolyzing or cellulolytic enzymes, require cations for their activity. Usually, mono- or divalent cations as NH₄⁺, K⁺, Na⁺ or Ca²⁺, Mn²⁺, Mg²⁺, Sr²⁺, Fe²⁺, Cu²⁺, Ni²⁺, Co²⁺, Zn²⁺ have to be added to obtain satisfying enzymatic activity. Thus, “activity at low addition of cations”, preferably activity without addition of cations, means that a polypeptide of the invention shows activity and is stable towards its environment conditions at a low cation concentration, preferably without addition of any cations at all.

“High specific activity” of the polypeptide of the invention means that its activity is essentially directed only towards its substrates.

“Functional”, e.g. functional fragment or functional derivative according to the invention means that the polypeptides exhibits cellulolytic activity towards their substrates, particularly any hydrolytic effect on cellulase substrates. Especially, it relates to the hydrolysis of the 3-D-glucosidic linkages of cellulase substrates, e.g. cellulose, carboxymethyl cellulose, cellulose polymers etc., to shorter cello-oligosaccharide oligomers, cellobiose and/or glucose. Several methods for measuring cellulolytic activity are known by a person skilled in the art (e.g. enzyme assays using marked substrates, substrate analysis by chromatographic methods (as HPLC or TLC) for separating enzyme and substrate and spectrophotometric assays for measuring hydrolytic activity) (see e.g., Sambrook J, Maniatis T (1989) Molecular Coning: A laboratory manual, Cold Spring Harbor Laboratory Press, Cold Spring Harbor, NY). Among various alternatives, an appropriate method is, for example, the cellulase activity assay, as described below (see Example 5). This assay system is based on an activity selection technique with Ostazin-brilliant red-hydroxyethyl cellulose as assay substrate.

The term “fragment of a polypeptide” according to the invention is intended to encompass a portion of a amino sequence disclosed herein of at least about 60 contiguous amino acids, preferably of at least about 80 contiguous amino acids, more preferably of at least about 100 contiguous amino acids or longer in length. Functional fragments which encode polypeptides that retain their activity are particularly useful.

A “derivative of a polypeptide” according to the invention is intended to indicate a polypeptide which is derived from the native polypeptide by substitution of one or more amino acids at one or two or more of different sites of the native amino acid sequence, deletion of one or more amino acids at either or both ends of the native amino acid sequence or at one or more sites of the amino acid sequence, or insertion of one or more amino acids at one or more sites of the native amino acid sequence retaining its characteristic activity, particularly cellulolytic activity. Such a polypeptide can possess altered properties which may be advantageous over the properties of the native sequence for certain applications (e.g. increased pH optimum, increased temperature stability etc.).

A derivative of a polypeptide according to the invention means a polypeptide which has substantial identity with the amino acid sequences disclosed herein. Particularly preferred are nucleic acid sequences which have at least 60% sequence identity, preferably at least 75% sequence identity, even more preferably at least 80%, yet more preferably 90% sequence identity and most preferably at least 95% sequence identity thereto. Appropriate methods for isolation of a functional derivative of a polypeptide as well as for determination of percent identity of two amino acid sequences is described below.

The production of such polypeptide fragments or derivatives (as described below) is well known and can be carried out following standard methods which are well known by a person skilled in the art (see e.g., Sambrook J, Maniatis T (1989) supra). In general, the preparation of such functional fragments or derivatives of a polypeptide can be achieved by modifying a DNA sequence which encode the native polypeptide, transformation of that DNA sequence into a suitable host and expression of the modified DNA sequence to form the functional derivative of the polypeptide with the provision that the modification of the DNA does not disturb the characteristic activity, particularly cellulolytic activity.

The isolation of these polypeptide fragments or derivatives (as described below) can be carried out using standard methods as separating from cell or culture medium by centrifugation, filtration or chromatography and precipitation procedures (see, e.g., Sambrook J, Maniatis T (1989) supra).

The polypeptide of the invention can also be fused to at least one second moiety. Preferably, the second or further moiety/moieties does not occur in the cellulase as found in nature. The at least second moiety can be an amino acid, oligopeptide or polypeptide and can be linked to the polypeptide of the invention at a suitable position, for example, the N-terminus, the C-terminus or internally. Linker sequences can be used to fuse the polypeptide of the invention with at least one other moiety/moieties. According to one embodiment of the invention, the linker sequences preferably form a flexible sequence of 5 to 50 residues, more preferably 5 to 15 residues. In a preferred embodiment the linker sequence contains at least 20%, more preferably at least 40% and even more preferably at least 50% Gly residues. Appropriate linker sequences can be easily selected and prepared by a person skilled in the art. Additional moieties may be linked to the inventive sequence, if desired. If the polypeptide is produced as a fusion protein, the fusion partner (e.g., HA, HSV-Tag, His6) can be used to facilitate purification and/or isolation. If desired, the fusion partner can then be removed from polypeptide of the invention (e.g., by proteolytic cleavage or other methods known in the art) at the end of the production process.

According to another embodiment of the invention a nucleic acid encoding a polypeptide of the invention (or a functional fragment or functional derivative thereof or a functional fragment or functional derivative of said nucleic acid is provided. Preferably, the nucleic acid comprises or consists of one of the nucleic acid sequences of FIGS. 9 to 16.

The nucleic acids of the invention can be DNA or RNA, for example, mRNA. The nucleic acid molecules can be double-stranded or single-stranded, single stranded RNA or DNA can be either the coding (sense) strand or the non-coding (antisense) strand. If desired, the nucleotide sequence of the isolated nucleic acid can include additional non-coding sequences such as non 3′- and 5′-sequences (including regulatory sequences, for example). All nucleic acid sequences, unless otherwise designated, are written in the direction from the 5′ end to the 3′ end.

Furthermore, the nucleic acids of the invention can be fused to a nucleic acid comprising, for example, a marker sequence or a nucleotide sequence which encodes a polypeptide to assist, e.g., in isolation or purification of the polypeptide. Representative sequences include, but are not limited to those which encode a glutathione-S-transferase (GST) fusion protein, a polyhistidine (e.g., His6), hemagglutinin, HSV-Tag, for example.

The term “nucleic acid” relates also to a fragment or derivative of said nucleic acid as described below.

The term “fragment of a nucleic acid” is intended to encompass a portion of a nucleotide sequence described herein which is from at least about 25 contiguous nucleotides to at least about 50 contiguous nucleotides, preferably at least about 60 contiguous nucleotides, more preferably at least about 120 contiguous nucleotides, most preferably at least about 180 contiguous nucleotides or longer in length. Especially, shorter fragments according to the invention are useful as probes and also as primer. Particularly preferred primers and probes selectively hybridize to the nucleic acid molecule encoding the polypeptides described herein. A primer is a nucleic acid fragment which functions as an initiating substrate for enzymatic or synthetic elongation. A probe is a nucleic acid sequence which hybridizes with a nucleic acid sequence of the invention, a fragment or a complementary nucleic acid sequence thereof. Fragments which encode polypeptides according to the invention that retain activity are particularly useful.

Hybridization can be used herein to analyze whether a given fragment or gene corresponds to the cellulase described herein and thus falls within the scope of the present invention. Hybridization describes a process in which a strand of nucleic acid joins with a complementary strand through base pairing. The conditions employed in the hybridization of two non-identical, but very similar, complementary nucleic acids varies with the degree of complementary of the two strands and the length of the strands. Such conditions and hybridisation techniques are well known by a person skilled in the art and can be carried out following standard hybridization assays (see e.g., Sambrook J, Maniatis T (1989) supra). Consequently, all nucleic acid sequences which hybridize to the nucleic acid or the functional fragments or functional derivatives thereof according to the invention are encompassed by the invention.

A “derivative of a nucleic acid” according to the invention is intended to indicate a nucleic acid which is derived from the native nucleic acid corresponding to the description above relating to a “functional derivative of a polypeptide”, i.e. by addition, substitution, deletion or insertion of one or more nucleic acids retaining the characteristic activity, particularly cellulolytic activity of said nucleic acid. Such a nucleic acid can exhibit altered properties in some specific aspect (e.g. increased or decreased expression rate).

As skilled artisans will recognize that the amino acids of polypeptides of the invention can be encoded by a multitude of different nucleic acid triplets because most of the amino acids are encoded by more than one nucleic acid triplet due to the degeneracy of the amino acid code. Because these alternative nucleic acid sequences would encode the same amino acid sequences, the present invention further comprises these alternate nucleic acid sequences.

A derivative of a nucleic acid according to the invention means a nucleic acid or a fragment or a derivative thereof which has substantial identity with the nucleic acid sequences described herein. Particularly preferred are nucleic acid sequences which have at least about 30%, preferably at least about 40%, more preferably at least about 50%, even more preferably at least about 60%, yet more preferably at least about 80%, still more preferably at least about 90%, and even more preferably at least about 95% identity with nucleotide sequences described herein.

To determine the percent identity of two nucleotide sequences, the sequences can be aligned for optimal comparison purposes (e.g., gaps can be introduced in the sequence of a first nucleotide sequence). The nucleotides at corresponding nucleotide positions can then be compared. When a position in the first sequence is occupied by the same nucleotide as the corresponding position in the second sequence, then the molecules are identical at that position. The percent identity between the two sequences is a function of the number of identical positions shared by the sequences.

The determination of percent identity of two sequences can be accomplished using a mathematical algorithm. A preferred, non-limiting example of a mathematical algorithm utilized for the comparison of two sequences is the algorithm of Karlin et al. (1993), PNAS USA, 90:5873-5877. Such an algorithm is incorporated into the NBLAST program which can be used to identify sequences having the desired identity to nucleotide sequences of the invention. To obtain gapped alignments for comparison purposes, Gapped BLAST can be utilized as described in Altschul et al. (1997), Nucleic Acids Res, 25:3389-3402. When utilizing BLAST and Gapped BLAST programs, the default parameters of the respective programs (e.g., NBLAST) can be used. The described method of determination of the percent identity of two can be also applied to amino acid sequences.

The production of such nucleic acid fragments or derivatives (as described below) is well known and can be carried out following standard methods which are well known by a person skilled in the art (see e.g., Sambrook J, Maniatis T (1989) supra). In general the preparation of such functional fragments or derivatives of a nucleic acid can be achieved by modifying (altering) a DNA sequence which encodes the native polypeptide and amplifying the DNA sequence with suitable means, e.g., by PCR technique. These mutations of the nucleic acids may be generated by either random mutagenesis techniques, such as those techniques employing chemical mutagens, or by site-specific mutagenesis employing oligonucleotides. These nucleic acids conferring substantially the same function, as described above, in substantially the same manner as the exemplified nucleic acids are also encompassed within the present invention.

Accordingly, derivatives of a polypeptide according to the invention (as described above) encoded by the nucleic acids of the invention may also be induced by alterations of the nucleic acids which encodes these proteins.

One of the most widely employed technique for altering a nucleic acid sequence is by way of oligonucleotide-directed site-specific mutagenesis (see Comack B, CURRENT PROTOCOLS IN MOLECULAR BIOLOGY, 8.01-8.5.9, Ausubel F, et al., eds. 1991). In this technique an oligonucleotide, whose sequence contains a mutation of interest, is synthesized as described supra. This olignucleotide is then hybridized to a template containing the wild-type nucleic acid sequence. In a preferred embodiment of this technique, the template is a single-stranded template. Particularly preferred are plasmids which contain regions such as the f1 intergenic region. This region allows the generation of single-stranded templates when a helper phage is added to the culture harboring the phagemid. After annealing of the oligonucleotide to the template, a DNA-dependent DNA polymerase is used to synthesize the second strand from the oligonucleotide, complementary to the template DNA. The resulting product is a heteroduplex molecule containing a mismatch due to the mutation in the oligonucleotide. After DNA replication by the host cell a mixture of two types of plasmid are present, the wild-type and the newly constructed mutant. This technique permits the introduction of convenient restriction sites such that the coding nucleic acid sequence may be placed immediately adjacent to whichever transcriptional or translational regulatory elements are employed by the practitioner.

The construction protocols utilized for E. coli can be followed to construct analogous vectors for other organisms, merely by substituting, if necessary, the appropriate regulatory elements using techniques well known to skilled artisans.

The isolation of such nucleic acid functional fragments or functional derivatives (as described below) can be carried out by using standard methods as screening methods (e.g., screening of a genomic DNA library) followed by sequencing or hybridisation (with a suitable probe, e.g., derived by generating an oligonucleotide of desired sequence of the “target” nucleic acid) and purification procedures, if appropriate.

The invention also relates to isolated nucleic acids. An “isolated” nucleic acid molecule or nucleotide sequence is intended to mean a nucleic aid molecule or nucleotide sequence which is not flanked by nucleotide sequences which normally flank the gene or nucleotide sequence (as in genomic sequences) and/or has been completely or partially purified from other nucleic acids (e.g., as in an DNA or RNA library). For example, an isolated nucleic acid of the invention may be substantially isolated with respect to the complex cellular milieu in which it naturally occurs. In some instances, the isolated material will form a part of a composition (for example, a crude extract con other substances), buffer system or reagent mix. In other circumstance, the material may be purified to essential homogeneity, for example as determined by PAGE or column chromatography such as HPLC. This meaning refers correspondingly to an isolated amino acid sequence.

The present invention also encompasses gene products of the nucleic acids of the invention coding for a polypeptide of the invention or a functional fragment or functional derivative thereof. Preferably the gene product codes for a polypeptide according to one of the amino acid sequences of FIG. 17 to 24. Also included are alleles, derivatives or fragments of such gene products.

“Gene product” according to the invention relates not only to the transcripts, accordingly RNA, preferably mRNA, but also to polypeptides or proteins, particularly, in purified form.

“Derivatives” or “fragments” of a gene product are defined corresponding to the definitions or derivatives or fragments of the polypeptide or nucleic acid according to the invention.

The invention also provides a vector comprising the nucleic acid of the invention. The terms “construct”, “recombinant construct” and “vector” are intended to have the same meaning and define a nucleotide sequence which comprises beside other sequences one or more nucleic acid sequences (or functional fragments, functional derivatives thereof) of the invention. A vector can be used, upon transformation into an appropriate host cell, to cause expression of the nucleic acid. The vector may be a plasmid, a phage particle or simply a potential genomic insert. Once transformed into a suitable host, the vector may replicate and function independently of the host genome, or may, under suitable conditions, integrate into the genome itself. Preferred vectors according to the invention are E. coli XL-Blue MRF and pBK-CMV plasmid.

The aforementioned term “other sequences” of a vector relates to the following: In general, a suitable vector includes an origin of replication, for example, Ori p, colEl Ori, sequences which allow the inserted nucleic acid to be expressed (transcribed and/or translated) and/or a selectable genetic marker including, e.g., a gene coding for a fluorescence protein, like GFP, genes which confer resistance to antibiotics such as the p-lactamase gene from Tn3, the kanamycin-resistance gene from Tn903 or the chloramphenicol-resistance gene from Tn9.

The term “plasmid” means an extrachromosomal usually self-replating genetic element. Plasmids are generally designated by a lower “p” preceded and/or followed by letters and numbers. The starting plasmids herein are either commercially available, publicly available on an unrestricted basis or can be constructed from available plasmids in accordance with the published procedures. In addition, equivalent plasmids to those described are known to a person skilled in the art. The starting plasmid employed to prepare a vector of the present invention may be isolated, for example, from the appropriate E. coli containing these plasmids using standard procedures such as cesium chloride DNA isolation.

A vector according to the invention also relates to a (recombinant) DNA cloning vector as well as to a (recombinant) expression vector. A DNA cloning vector refers to an autonomously replicating agent, including, but not limited to, plasmids and phages, comprising a DNA molecule to which one or more additional nucleic acids of the invention have been added. An expression vector relates to any DNA cloning vector recombinant construct comprising a nucleic acid sequence of the invention operably linked to a suitable control sequence capable of effecting the expression and to control the transcription of the inserted nucleic acid of the invention in a suitable host. Also, the plasmids of the present invention may be rely modified to construct expression vectors that produce the polypeptides of the invention in a variety of organisms, including for example, E. coli, Sf9 (as host for baculovirus), Spodoptera and Saccharomyces. The literature contains techniques for constructing AV12 expression vectors and for transforming AV12 host cells. U.S. Pat. No. 4,992,373, herein incorporated by reference, is one of many references describing these techniques.

“Operably linked” means that the nucleic acid sequence is linked to a control sequence in a manner which allows expression (e.g., transcription and/or translation) of the nucleic acid sequence.

“Transcription” means the process whereby information contained in a nucleic acid sequence of DNA is transferred to complementary RNA sequence.

“Control sequences” are well known in the art and are selected to express the nucleic acid of the invention and to control the transcription. Such control sequences include, but are not limited to a polyadenylation signal, a promoter (e.g., natural or synthetic promoter) or an enhancer to effect transcription, an optional operator sequence to control transcription, a locus control region or a silencer to allow a tissue-specific transcription, a sequence encoding suitable ribosome-binding sites on the mRNA, a sequence capable to stabilize the mRNA and sequences that control termination of transcription and translation. These control sequences can be modified, e.g., by deletion, addition, insertion or substitution of one or more nucleic acids, whereas saving their control function. Other suitable control sequences are well known in the art and are described, for example, in Goeddel (1990), Gene Expression Technology: Methods in Enzymology 185, Academic Press, San Diego, Calif.

Especially a high number of different promoters for different organism is known. For example, a preferred promoter for vectors used in Bacillus subtilis is the AprE promoter, a preferred promoter used in E. coli, is the T7/Lac promoter, a preferred promoter used in Saccharomyces cerevisiae is PGK1, a preferred promoter used in Aspergillus niger is glaA, and a preferred promoter used in Trichoderma reesei (reesei) is cbhI. Promoters suitable for use with prokaryotic hosts also include the beta-lactamase (vector pGX2907 (ATCC 39344) containing the replicon and beta-lactamase gene) and lactose promoter systems (Chang et al. (1978), Nature (London), 275:615; Goeddel et al. (1979), Nature (London), 281:544), alkaline phosphatase, the tryptophan (trp) promoter system (vector pATH1 (ATCC 37695) designed to facilitate expression of an open reading frame as a trpE fusion protein under control of the trp promoter) and hybrid promoters such as the tac promoter (isolatable from plasmid pDR540 ATCC-37282). However, other functional bacterial promoters, whose nucleotide sequences are generally known, enable a person skilled in the art to ligate them to DNA encoding the polypeptides of the instant invention using linkers or adapters to supply any required restriction sites. Promoters for use in bacterial systems also will contain a Shine-Dalgarno sequence operably linked to the DNA encoding the desired polypeptides.

Useful expression vectors, for example, may consist of segments of chromosomal, non-chromosomal and synthetic DNA sequences such as various known derivatives of SV40 and known bacterial plasmids, e.g., plasmids from E. coli including col E1, pBK, pCR1, pBR322, pMb9, pUC 19 and their derivatives, wider host range plasmids, e.g., RP4, phage DNAs e.g., the numerous derivatives of phage lambda, e.g., NM989, and other DNA phages, e.g., M13 and filamentous single stranded DNA phages, yeast plasmids, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present invention are known in the art and are described generally in, for example, Sambrook J, Maniatis T (1989) supra.

The invention also provides a host cell comprising a vector or a nucleic acid (or a functional fragment, or a functional derivative thereof according to the invention.

“Host cell” means a cell which has the capacity to act as a host and expression vehicle for a nucleic acid or a vector according to the present invention. The host cell can be e.g., a prokaryotic, an eukaryotic or an archaeon cell. Host cells comprising (for example, as a result of transformation, transfection or transduction) a vector or nucleic acid as described herein include, but are not limited to, bacterial cells (e.g., R. marinus, E. coli, Streptomyces, Pseudomonas, Bacillus, Serratia marcescens, Salmonella typhimurium), fungi including yeasts (e.g., Saccharomyces cerevisie, Pichia pastoris) and molds (e.g., Aspergillus sp.), insect cells (e.g., Sf9) or mammalian cells (e.g., COS, CHO). In a preferred embodiment according to the present invention, host cell means the cells of E. coli.

Eukaryotic host cells are not limited to use in a particular eukaryotic host cell. A variety of eukaryotic host cells are available, e.g., from depositories such as the American Type Culture Collection (ATCC) and are suitable for use with the vectors of the present invention. The choice of a particular host cell depends to some extent on the particular expression vector used to drive expression of the nucleic acids of the present invention. Eukaryotic host cells include mammalian cells as well as yeast cells.

The imperfect fungus Saccharomyces cerevisiae is the most commonly used eukaryotic microorganism, although a number of other strains are commonly available. For expression in Saccharomyces sp., the plasmid YRp7 (ATCC-40053), for example, is commonly used (see. e.g., Stinchcomb L. et al. (1979) Nature, 282:39; Kingsman J. al. (1979), Gene, 7:141; S. Tschemper et al. (1980), Gene, 10:157). This plasmid already contains the trp gene which provides a selectable marker for a mutant stain of yeast lacking the ability to grow in tryptophan.

Suitable promoting sequences for use with yeast hosts include the promoters for 3-phosphoglycerate kinase (found on plasmid pAP12BD (ATCC 53231) and described in U.S. Pat. No. 4,935,350, issued Jun. 19, 1990, herein incorporated by reference) or other glycolytic enzymes such as enolase (found on plasmid pAC1 (ATCC 39532)), glyceraldehyde-3-phosphate dehydrogenase (derived from plasmid pHcGAPC1 (ATCC 57090, 57091)), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase, as well as the alcohol dehydrogenase and pyruvate decarboxylase genes of Zymomonas mobilis (U.S. Pat. No. 5,000,000 issued Mar. 19, 1991, herein incorporated by reference).

Other yeast promoters, which are inducible promoters, having the additional advantage of their transcription being controllable by varying growth conditions, are the promoter regions for alcohol dehydrogenase 2, isocytochrome C, acid phosphatase, degradative enzymes associated with nitrogen metabolism, metallothionein (contained on plasmid vector pCL28XhoLHBPV (ATCC 39475) and described in U.S. Pat. No. 4,840,896, herein incorporated by reference), glyceraldehyde 3-phosphate dehydrogenase, and enzymes responsible for maltose and galactose (e.g. GAL1 found on plasmid pRY121 (ATCC 37658)) utilization. Yeast enhancers such as the UAS Ga1 from Saccharomyces cerevisiae (found in conjunction with the CYC1 promoter on plasmid YEpsec-hI1beta ATCC 67024), also are advantageously used with yeast promoters.

A vector can be introduced into a host cell using any suitable method (e.g., transformation, electroporation, transfection using calcium chloride, rubidium chloride, calcium phosphate, DEAEdextran or other substances, microprojectile bombardment, lipofection, infection or transduction). Transformation relates to the introduction of DNA into an organism so that the DNA is replicable, either as an extrachromosomal element or by chromosomal integration. Methods of transforming bacterial and eukaryotic hosts are well known in the art. Numerous methods, such as nuclear injection, protoplast fusion or by calcium treatment are summerized in Sambrook J, Maniatis T (1989) supra. Transfection refers to the taking up of a vector by a host cell whether or not any coding sequences are in fact expressed. Successful transfection is generally recognized when any indication or the operation or this vector occurs within the host cell.

Another embodiment of the invention provides a method for the production of the polypeptide of the invention comprising the following steps:
(a) cultivating a host cell of the invention and expressing the nucleic acid under suitable conditions;
(b) isolating the polypeptide with suitable means.

The polypeptides according to the present invention may also be produced by recombinant methods. Recombinant methods are preferred if a high yield is desired. A general method for the construction of any desired DNA sequence is provided, e.g., in Brown J. et al. (1979), Methods in Enzymology, 68:109; Sambrook J, Maniatis T (1989), supra.

According to the invention an activity-based screening in metagenome library was used as a powerful technique to isolated new enzymes from the big diversity of microorganisms found in rumen ecosystem (Lorenz, P et al. (2002) Screening for novel enzymes for biocatalytical processes:accessing the metagenome as a resource of novel functional sequence space, Current Opinion in Biotechnology 13:572-577). For this purposes an activity selection technique with Ostazin-brilliant red-hydroxyethyl cellulose as assay substrate, was used. This technology enable screening of 105-109 clones/day from genomes of between 1 and 15,000 microorganisms. In detail, this method is described in the Examples below.

The polypeptide can be isolated from the culture medium by conventional procedures including separating the cells from the medium by centrifugation or filtration, if necessary after disruption of the cells, precipitating the proteinaceous components of the supernatant or filtrate by means of a salt, e.g., ammonium sulfate, followed by purification by a variety of chromatographic procedures, e.g., ion exchange chromatography, affinity chromatography or similar art recognized procedures.

Efficient methods for isolating the polypeptide according to the present invention also include to utilize genetic engineering techniques by transforming a suitable host cell with a nucleic acid or a vector provided herein which encodes the polypeptide and cultivating the resultant recombinant microorganism, preferably E. coli, under conditions suitable for host cell growth and nucleic acid expression, e.g., in the presence of inducer, suitable media supplemented with appropriate salts, growth factors, antibiotic, nutritional supplements, etc.), whereby the nucleic acid is expressed and the encoded polypeptide is produced.

In additional embodiments the polypeptide of the invention can be produced by in vitro translation of a nucleic acid that encodes the polypeptide, by chemical synthesis (e.g., solid phase peptide synthesis) or by any other suitable method.

Skilled artisans will recognize that the polypeptides of the present invention can also be produced by a number of different methods. All of the amino acid sequences of the invention can be made by chemical methods well known in the art, including solid phase peptide synthesis, or recombinant methods. Both methods are described in U.S. Pat. No. 4,617,149, the entirety of which is herein incorporated by reference.

The principles of solid phase chemical synthesis of polypeptides are well known in the art and are described by, e.g., Dugas H. and Penney C. (1981), Bioorganic Chemistry, pages 54-92. For examples, peptides may be synthesized by solid-phase methodology utilizing an Applied Biosystems 430A peptide synthesizer (commercially available from Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

Sequential t-butoxycarbonyl chemistry using double couple protocols are applied to the starting p-methyl benzhydryl amine resins for the production of C-terminal carboxamides. For the production of C-terminal acids, the corresponding pyridine-2-aldoxime methiodide resin is used. Asparagine, glutamine, and arginine are coupled using preformed hydroxy benzotriazole esters. The following side chain protection may be used:

### Arg, Tosyl

Asp, cyclohexyl
Glu, cyclohexyl

### Ser, Benzyl

### Thr, Benzyl

Tyr, 4-bromo carbobenzoxy

Removal of the t-butoxycarbonyl moiety (deprotection) may be accomplished with trifluoroacetic acid (TFA) in methylene chloride. Following completion of the synthesis the peptides may be deprotected and cleaved from the resin with anhydrous hydrogen fluoride containing 10% meta-cresol. Cleavage of the side chain protecting group(s) and of the peptide from the resin is carried out at zero degrees centigrade or below, preferably −20° C. for thirty minutes followed by thirty minutes at 0° C.

After removal of the hydrogen fluoride, the peptide/resin is washed with ether, and the peptide extracted with glacial acetic acid and then lyophilized. Purification is accomplished by size-exclusion chromatography on a Sephadex G-10 (Pharmacia) column in 10% acetic acid.

Another embodiment of the invention relates to the use of the polypeptide, the nucleic acid, the vector and/or the host cell of the invention (hereinafter “substances of the invention”) for the treatment of cellulosic textiles or fabrics, e.g. as an ingredient in compositions, preferably detergent compositions or fabric softener compositions. Consequently, the invention relates also to detergent compositions including a polypeptide according to the invention.

Cellulases and thus substances of the invention can be used for modification of a broad range of cellulose containing textile material. It opens also the possibility to make use of cellulase technology for introduction of subtle decorative patterns on dyed cloth. This result in more bright colors at those treated places and thus subtle color differences following the pattern. Examples of the uses of substances of the invention can also be found in the denim garment washing industry to give a stone washed look to denim without the use of pumice stones and in the textile finishing industry to give the cloth a smooth surface with improved wash and wear resistance and a softer hand (biopolishing).

The treatment of cellulosic textiles or fabrics includes textile processing or cleaning with a composition comprising a polypeptide of to the present invention. Such treatments include, but are not limited to, stonewashing, modifying the texture, feel and/or appearance of cellulose containing fabrics or other techniques used during manufacturing or cleaning/reconditioning of cellulose containing fabrics. Additionally, treating within the context of this invention contemplates the removal of immature cotton from cellulosic fabrics or fibers.

The detergent resistance or in other words the effect of surfactants on the activity of the polypeptide of the invention was measured. The results are given in FIG. 3 and show that the activity of the polypeptide is increased in the presence of Triton X-100 and SDS whereas other surfactants, like Tween 20-80, polyethylene alkyl ether did not affect the activity of the polypeptides. Also, the effect of several commonly used inhibitors on the activity of the polypeptide of the invention was investigated. Sulfhydryl inhibitors such as N-ethylmaleimide, iodoacetate and p-chloromercuribenzoate were all inhibitory. The chelating agents EDTA (ethylenediaminetetraacetic acid) and EGTA (ethyleneglycoltetraacetic acid) a more efficient chelator of Ca²⁺ (inhibition >30%) and O-phenanthroline (inhibition >71%) had essentially no effect on the activity of the polypeptides or were slightly stimulatory. In summary, the polypeptides of the invention are well suited to be used for the treatment of cellulosic textiles or fabrics, especially as an ingredient in a detergent composition, because of their high activity at high pH and high stability against metal ions or various components of laundry products such as surfactants, chelating agents or proteinases. In general, they are also suited to be used in compositions containing several commonly used inhibitors.

Thus, substances of the present invention can be employed in a detergent composition. Such a detergent compositions is useful as pre-wash compositions, pre-soak compositions, or for cleaning during the regular wash or rinse cycle. Preferably, the detergent compositions of the present invention comprise an effective amount of a substance of the invention, surfactants, builders, electrolytes, alkalis, antiredeposition agents, bleaching agents, antioxidants, solubilizer and other suitable ingredients known in the art.

An “effective amount” of polypeptide employed in the detergent compositions of this invention is an amount sufficient to impart the desirable effects and will depend on the extent to which the detergent will be diluted upon addition to water so as to form a wash solution.

“Surfactants” of the detent composition can be anionic (e.g., linear or branche alkylbenzenesulfonates, alkyl or alkenyl ether sulfates having linear or branche alkyl groups or alkenyl groups, alkyl or alkenyl sulfates, olefinsulfonates and alkanesulfonates), ampholytic (e.g., quaternary ammonium salt sulfonates and betaine-type ampholytic surfactants) or non-ionic surfactants (e.g., polyoxyalkylene ethers, higher fatty acid alkanolamides or alkylene oxide adduct thereof fatty acid glycerine monoesters). It is also possible to use mixtures of such surfactants.

“Builders” of the detergent composition include, but are not limited to alkali metal salts and alkanolamine salts of the following compounds: phosphates, phosphonates, phosphonocarboxylates, salts of amino acids, aminopolyacetates high molecular electrolytes, non-dissociating polymers, salts of dicarboxylic acids, and aluminosilicate salts.

“Electrolytes” or “alkalis” of the detergent composition include, for example, silicates, carbonates and sulfates as well as organic alkalis such as triethanolamine, diethanolamine, monoethanolamine and triisopropanolamine.

“Antiredeposition agents” of the detergent composition include, for example, polyethylene glycol, polyvinyl alcohol, polyvinylpyrrolidone and carboxymethylcellulose.

“Bleaching agents” of the detergent composition include, for example, potassium monopersulfate, sodium percarbonate, sodium perborate sodium sulfate/hydrogen peroxide adduct and sodium chloride/hydrogen peroxide adduct or/and a photo-sensitive bleaching dye such as zinc or aluminum salt of sulfonated phthalocyanine further improves the detergenting effects.

“Antioxidants” of the detergent composition include, for example, tert-butyl-hydroxytoluene, 4,4′-butylidenebis (6tert-butyl-3-methylphenol), 2,2′-butylidenebis (6-tert-butyl-4-methylphenol), monostyrenated cresol, distyrenated cresol, monostyrenated phenol, distyrenated phenol and 1,1-bis(4hydroxy-phenyl)cyclohexane.

“Solubilizer” of the detergent composition include, for example, lower alcohols (e.g., ethanol), benzenesulfonate salts, lower alkylbenzenesulfonate salts (e.g., p-toluenesulfonate salts), glycols (e.g., propylene glycol), acetylbenzene-sulfonate salts, acetamides, pyridinedicarboxylic acid amides, benzoate salts and urea.

The detent compositions of the present invention may be in any suitable form, for example, as a liquid, in granules, in mulsions, in gels, or in pastes. Such forms are well known in the art and are described e.g., in U.S. Pat. No. 5,254,283 which is incorporated herein by reference in its entirety.

Further embodiments of the invention of the invention relate to the use of the substances of the invention in the treatment of pulp and paper industry, in consumer products, particularly food products and as an additive for animal food for purposes known in the art.

For example, cellulases, and therefore polypeptides of the invention, are known improve the drainability of wood pulp or paper pulp, to increase the value of animal food, enhance food products and reduce fiber in grain during the grain wet milling process or dry milling process.

Thus, substances of the invention can be applied in bakery to improve, for example, bread volume. Supplementation of polypeptides of the invention is useful to increase the total tract digestibility of organic matter and fiber during animal feeding. The proportion of the diet to which substances of the invention are applied must be maximized to ensure a beneficial response. For example, it is known that small proportion of cellulases on the diet increase the microbial N synthesis for cows.

Furthermore, substances of the invention can be applied to improve the use of cellulosic biomass and may offer a wide ran of novel applications in research, medicine and industry. One example is the production of biodiesel. Cellulases (and thus, substances of the invention) are able to hydrolyse cellulolytic substrate to more hydrolysable sugar which can be used as raw material for ethanol production. Related to medicine they could be applied for the destruction of pathogenic biofilm which are protected for the environment though a polysaccharide cover. They could help for its degradation, making them more accessible for antibiotic treatment. Cellulases (and thus, substances of the invention) are useful as a possible alternative to the current practice of open air burning of sugarcane residue by farmers. They are also useful as a chiral stationary phase in liquid chromatographic separations of enantiomers. Cellulases (and thus, substances of the invention) are also useful in the synthesis of transglycosilation product using cellulolytic substrate as raw materials. The transglycosylated product can be use for their beneficial effect in health as food additive, similar to FOS and GOS (probiotics).

Another embodiments of the invention relates to the use of the substances of the invention as a protoplast preparation agent, a herbicide or a drench in ruminants. Yet another embodiment of the invention relates to the use of the substances of the invention in the production of anti-cellulase reagents, e.g., bacteriocide and fungicide.

The substances of the invention can be expressed, for example, in plants.

The treatment according to the invention also comprises preparing an aqueous solution which contains an effective amount of the polypeptide of the invention together with other optional ingredients, for example, a surfactant, as described above, a scouring agent and/or a buffer. A buffer can be employed to maintain the pH of the aqueous solution within the desired range. Such suitable buffers are well known in the art. As described above, an effective amount of the polypeptide will depend on the intended purpose of the aqueous solution.

The following figures and examples are thought to illustrate the invention and should not be constructed to limit the scope of the invention thereon. All references cited by the disclosure of the present application are hereby incorporated in their entirety by reference.

### FIGURES

FIG. 1 shows Table 1 representing substrate specificity of rumen endo-beta-1,4-glucanases for various substrates. The values for clones pBKRR.1, 3, 4, 7, 9, 11, 13, 14, 16, 19 and 22 are depicted. The activity is expressed in μmol min⁻¹ g⁻¹ of E. coli lyophilised cells.

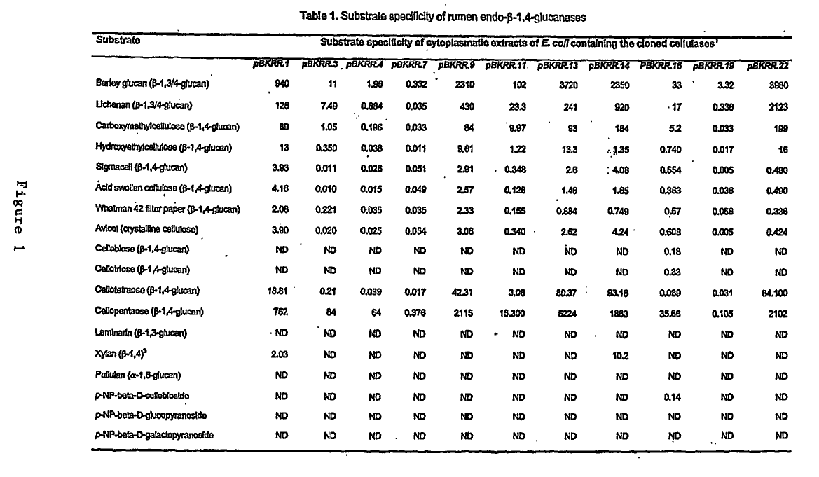

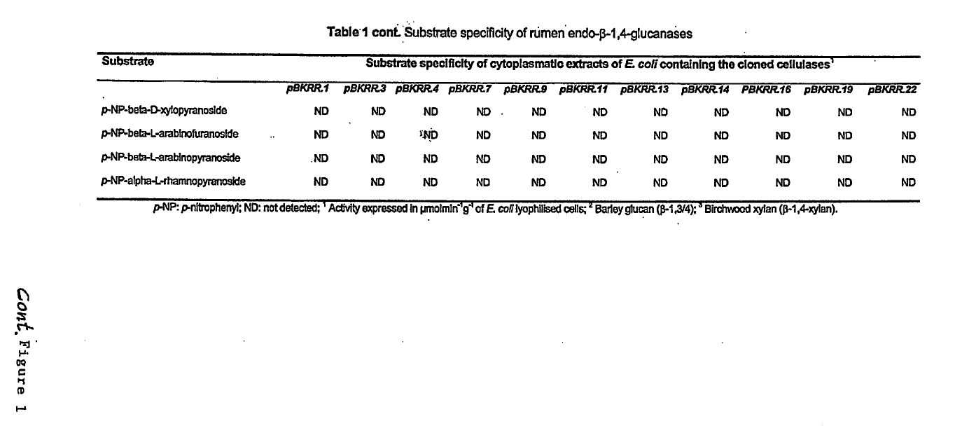

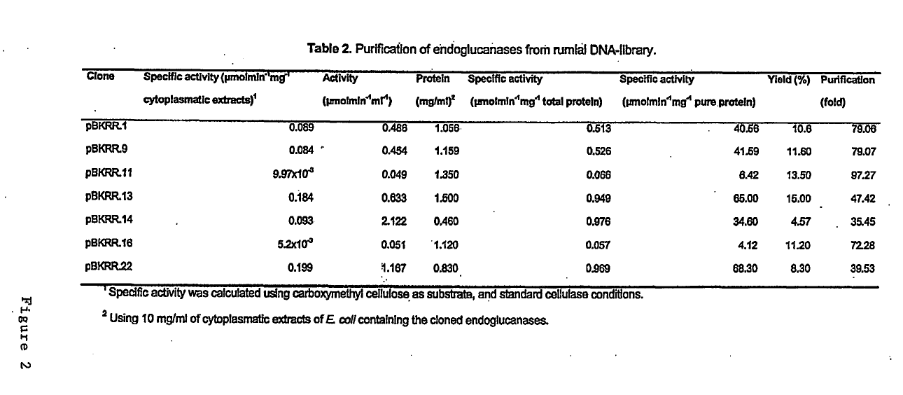

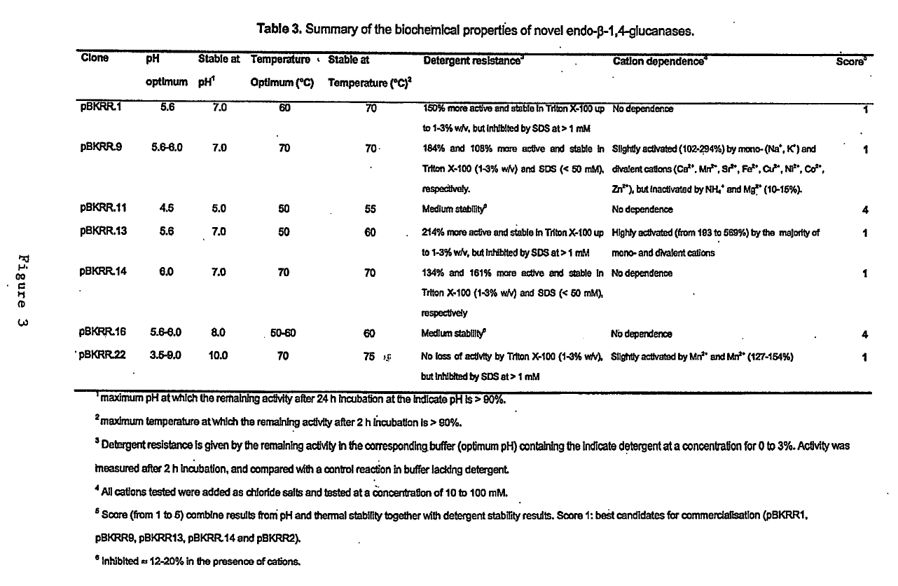

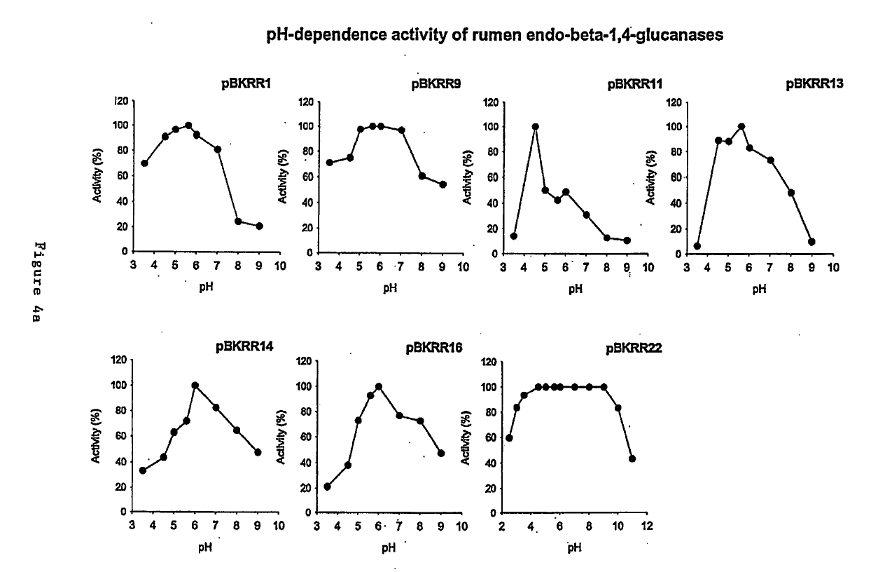

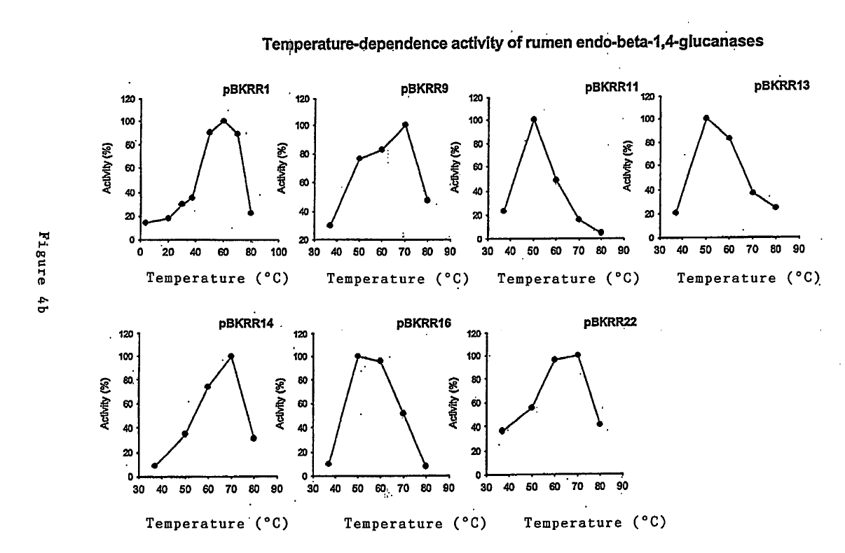

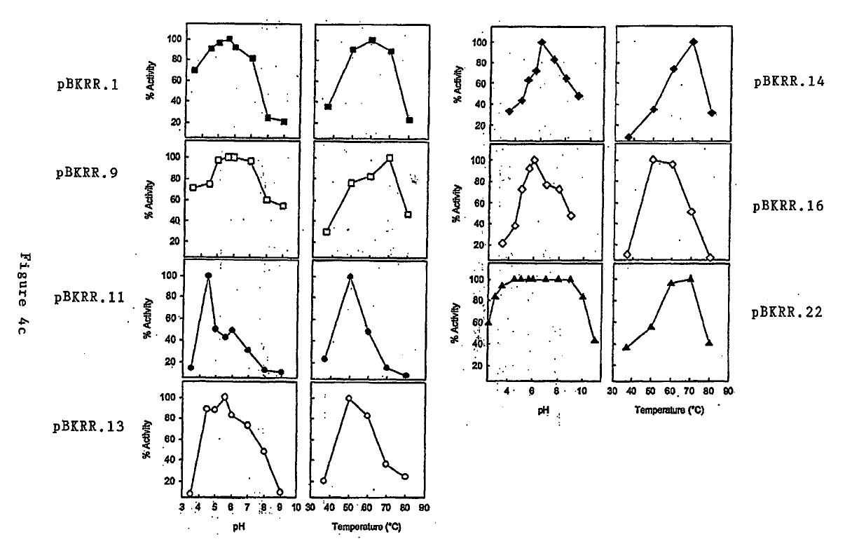

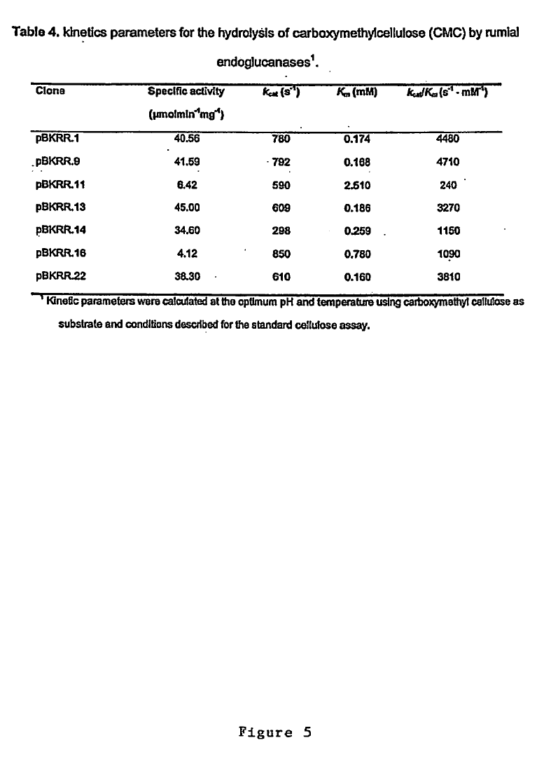

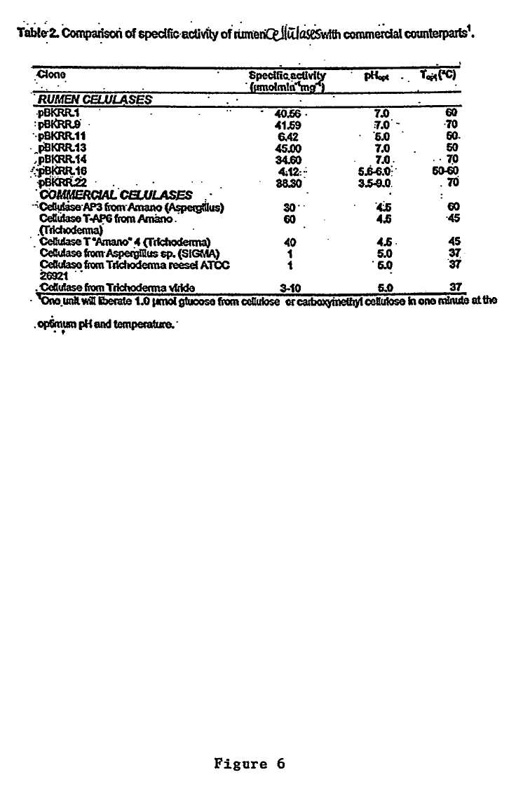

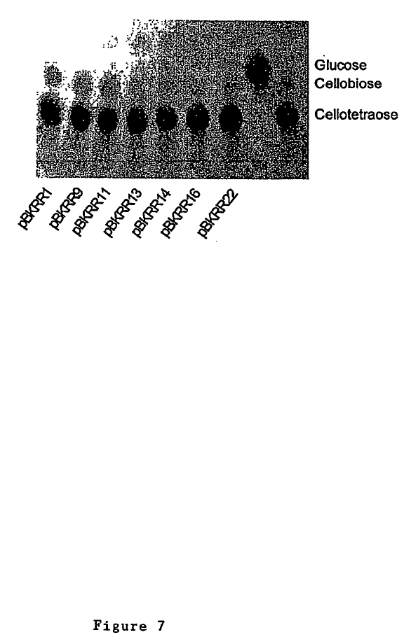

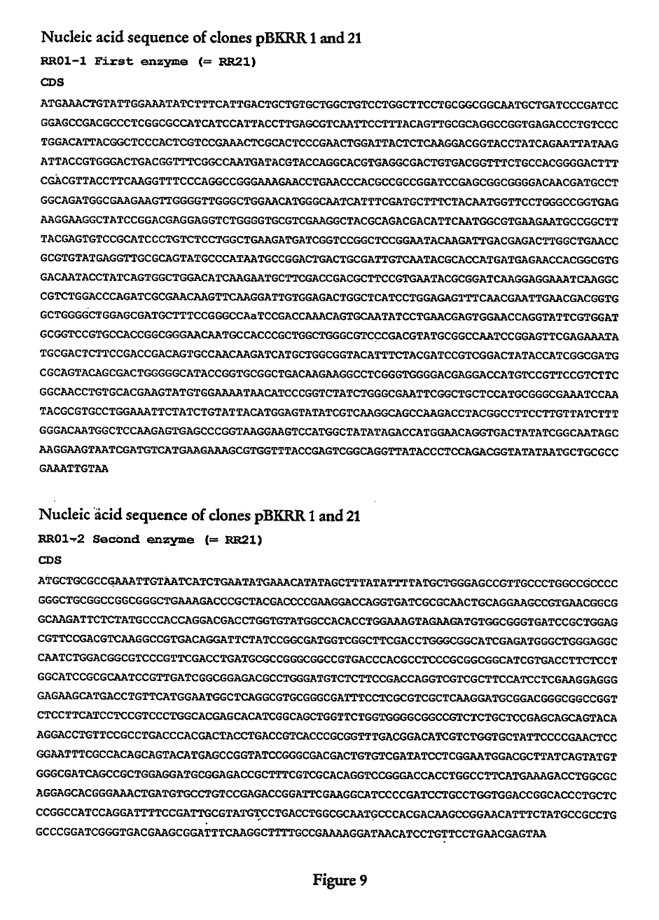

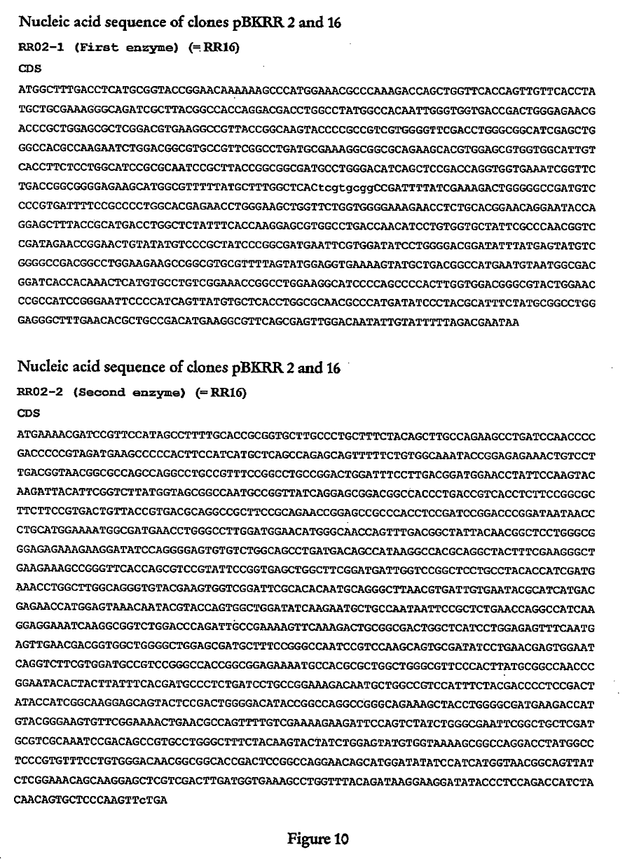

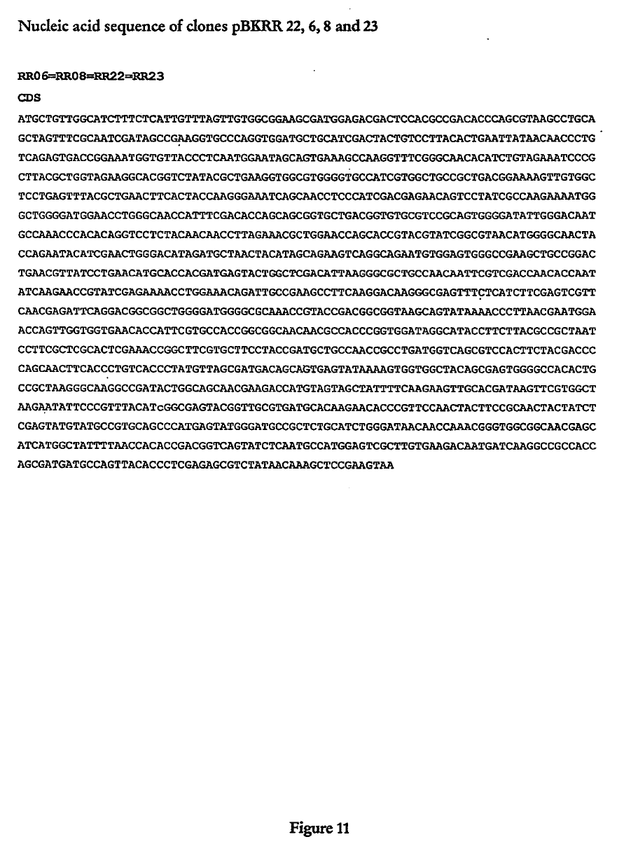

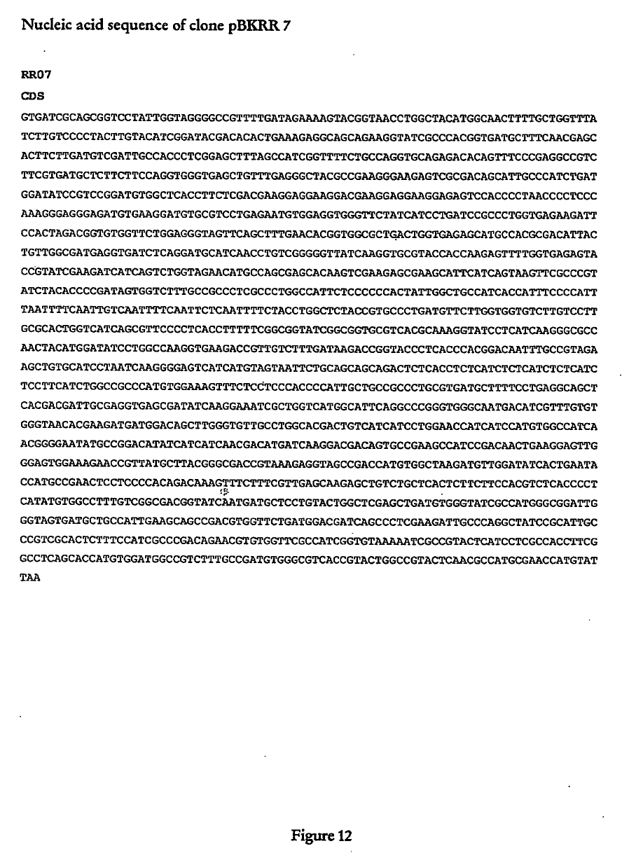

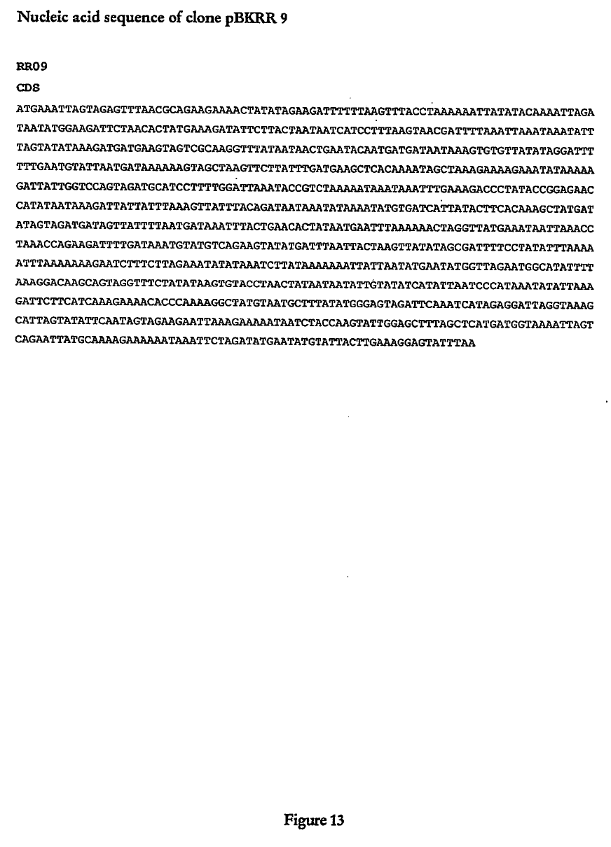

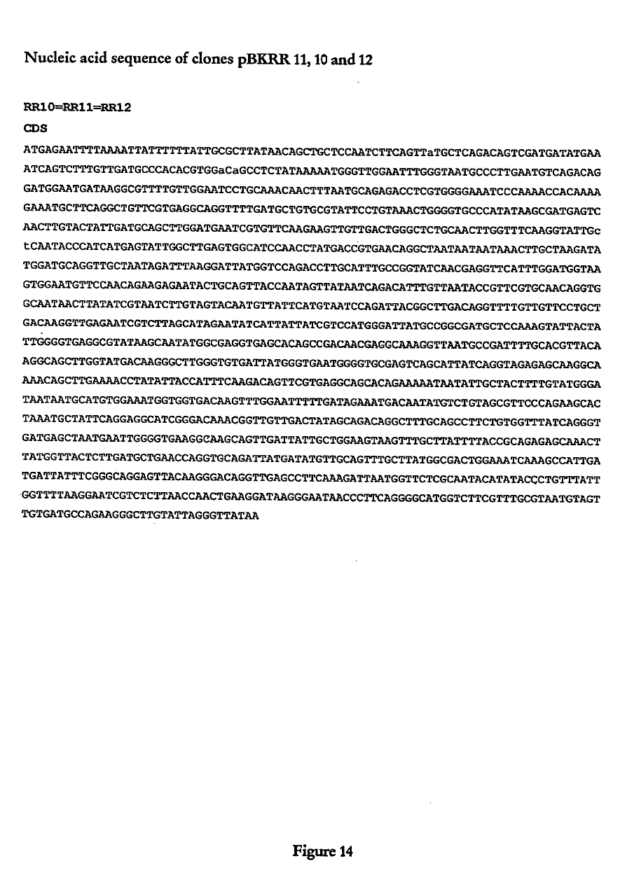

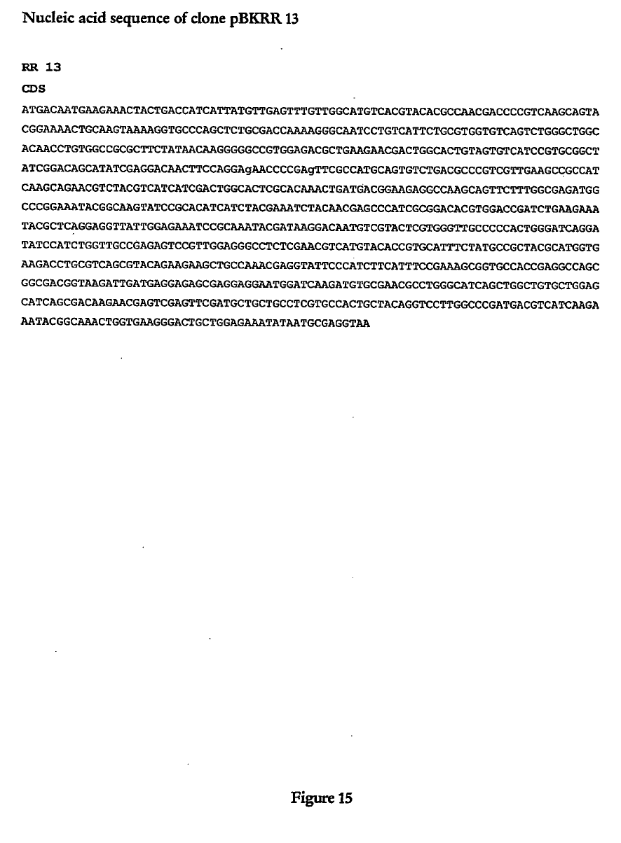

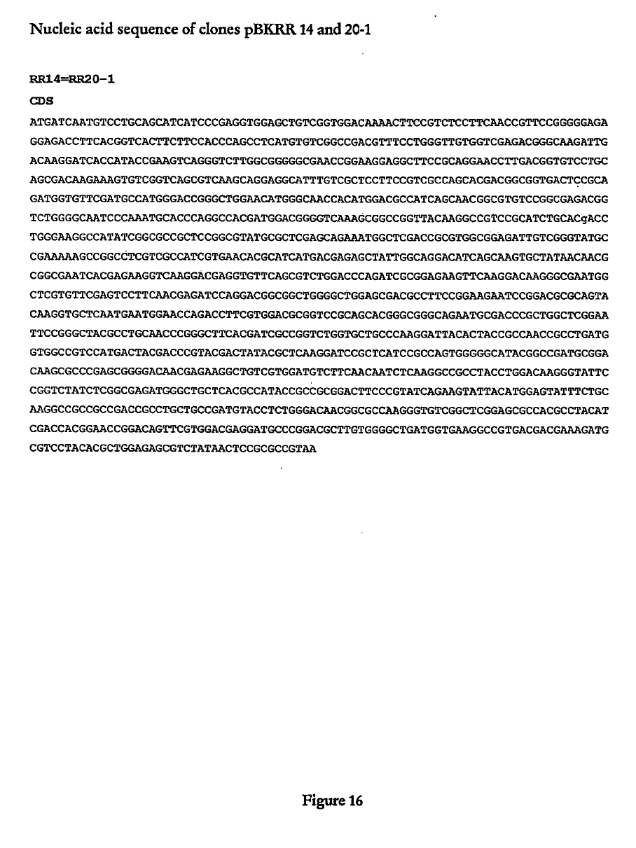

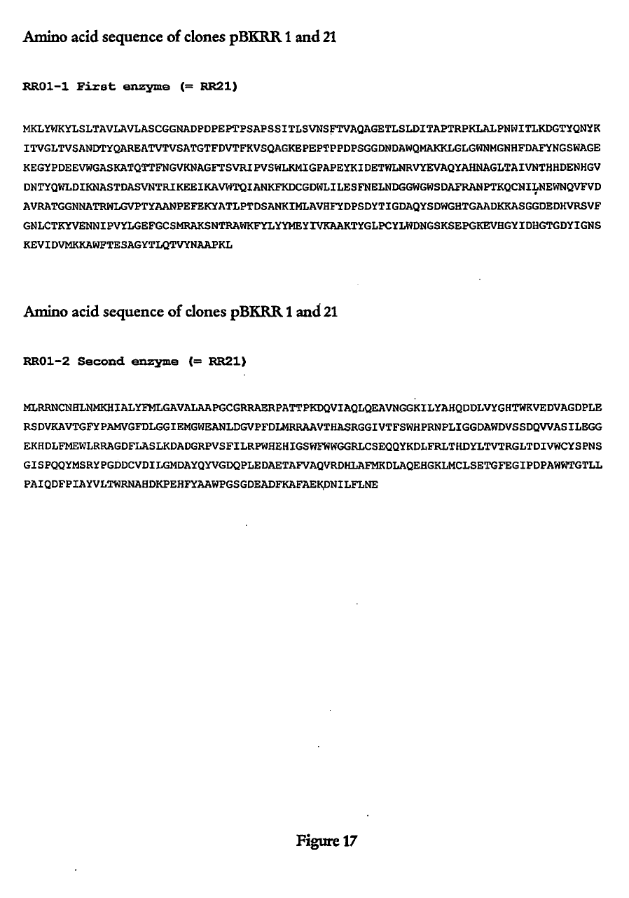

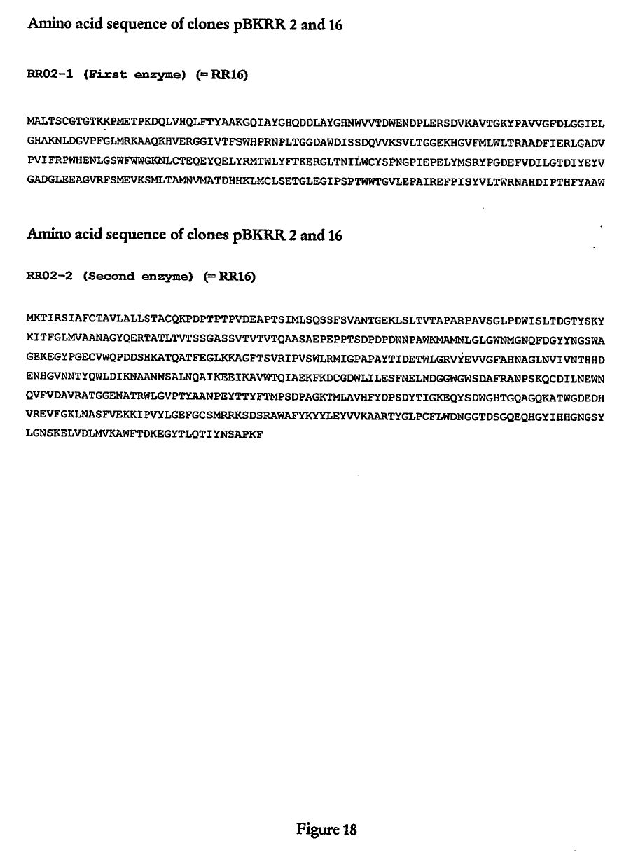

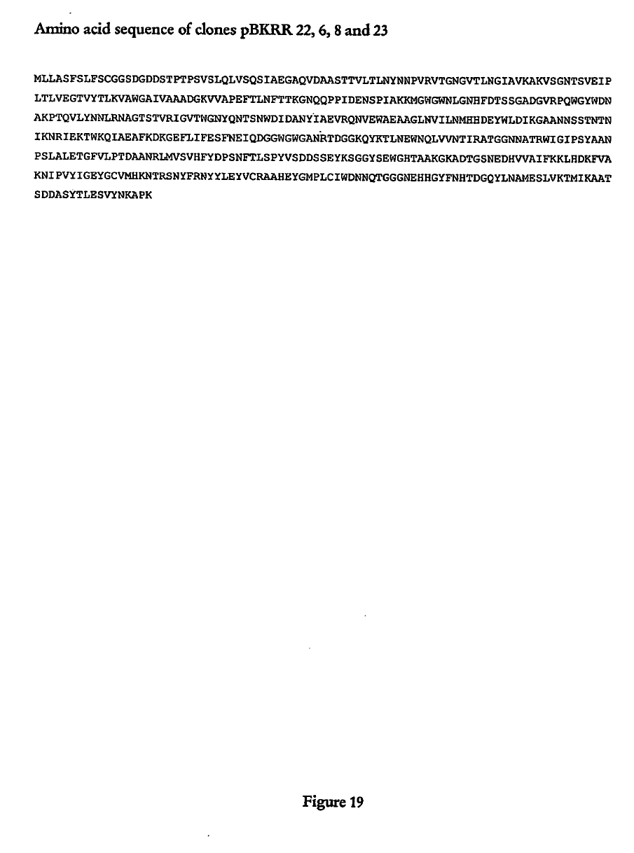

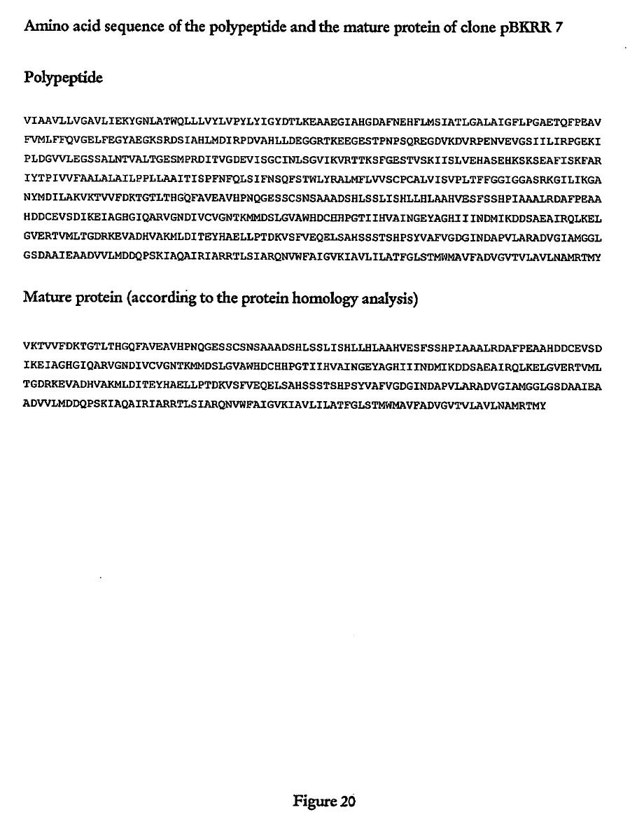

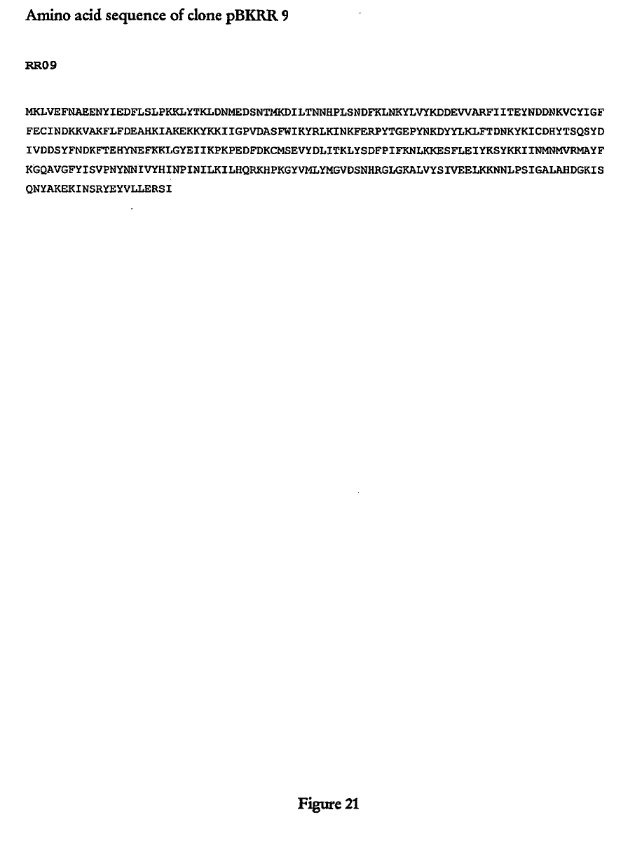

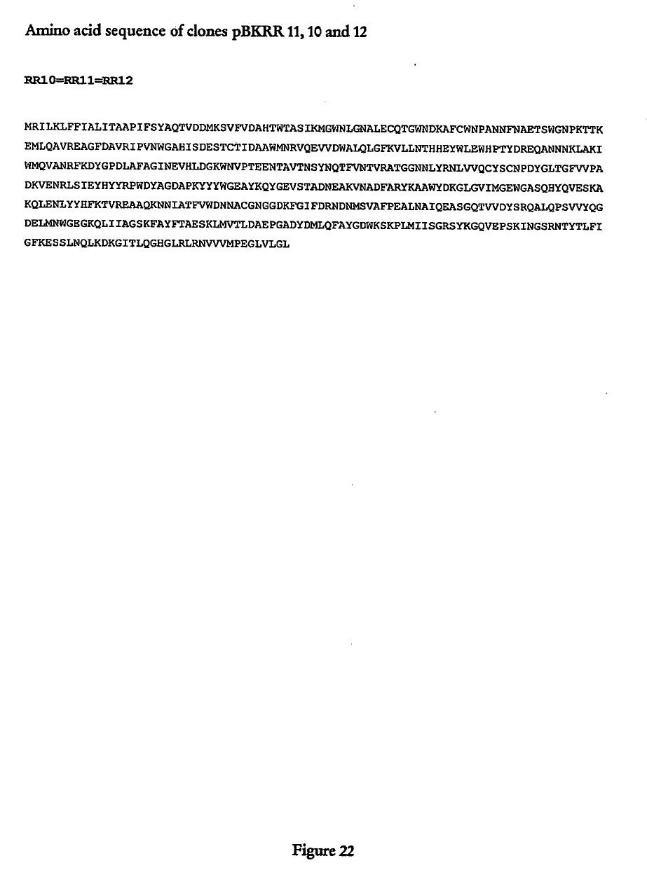

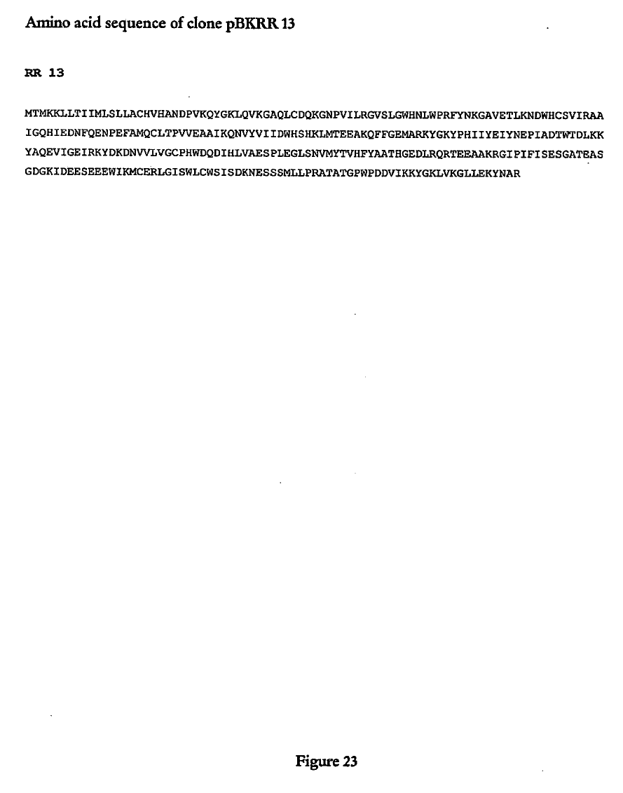

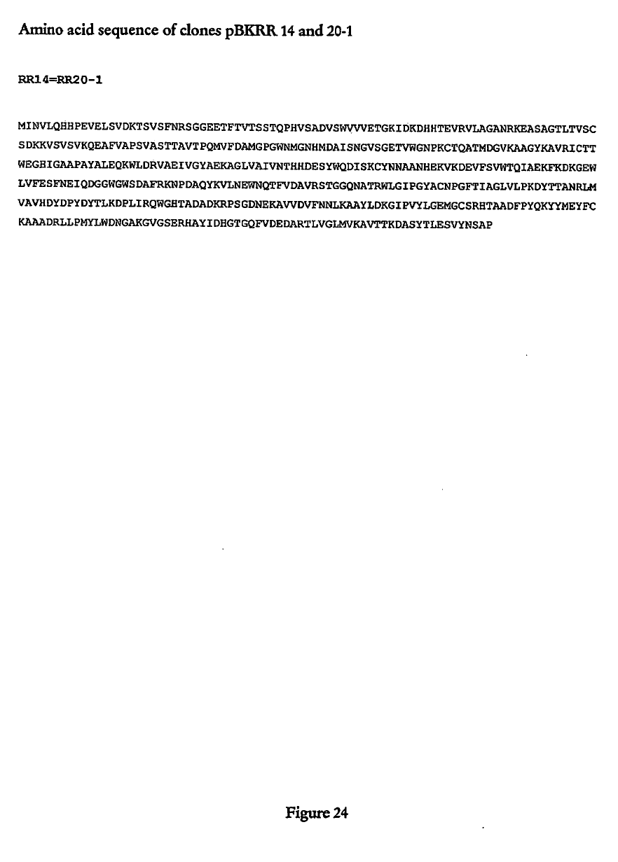

Endoglucanases were expressed in Escherichia coli under the control of P_{lac}-promoter. The relative level of cellulase expression was found to be pBKRR.22>pBKRR.14>pBKRR.1>pBKRR.9>pBKRR.13>pBKRR.3>pBKRR.4>pBKRR.7>pBKRR.11>pBKRR.16, and was in the range from 5 to 0.4% (w/w of total proteins). A preliminary quantitative assessment of enzymatic activities was performed by testing E. coli lyophilized cells bearing cellulose cDNAs, for their ability to hydrolyze various cellulose substrates (4% wt/vol), including glucan oligosaccharides with beta-1,4 backbone linkage (cellobiose, cellotriose, cellotetraose, cellopentose) or beta-1,3 linkage (laminarin), glucan polysaccharides, i.e. Barley glucan (beta-1,3/4), Lichenan (beta-1,3/4) and carboxymethyl cellulose (CMC), hydroxyethyl cellulose, Sigmacell®, acid swollen cellulose or filter paper (beta-1,4) as well as Avicel (crystalline cellulose), birchwood xylan (beta-1,4), and solubles p-nitrophenyl derivatives.

Results show that E. coli extracts expressing cellulose cDNAs had the highest activity towards cellulose oligosaccharides, specially (1,3),(1,4)-beta-glucans (from 0.3 to 3980 μmol min⁻¹ g⁻¹ of E. coli cells), such as barley beta-glucan and lichenan, similar to other endoglucanases found in 12 different families (Bauer et al., 1999, An Endoglucanase, EglA, from the Hyperthermophilic Archaeon Pyrococcus furiosus hydrolyzes beta-1,4 Bonds in Mixed-Linkage (1→3),(1→4)-beta-D-Glucans and Cellulose J. Bactriol., 181: 284-290). pBKRR.1, pBKRR.9, pBKRR.13, pBKRR.14 and pBKRR.22 had also significant activity towards substituted cellulose-based substrates, with only beta-1,4 linkages, such as CMC (from 89 to 199 μmol min⁻¹ g⁻¹ of lyophilized cells). pBKRR.11 and pBKRR.16 (from 5.2 to 9.9 μmol min⁻¹ g⁻¹), and in less extension pBKRR.3, pBKRR.4, pBKRR.7 and pBKRR.19 (from 0.19 to 1.05 μmol min⁻¹ g⁻¹) were able to hydrolyze CMC. pBKRR.16 hydrolyzed to a certain extent cellobiose and cellotriose as well as p-nitrophenyl cellobiose. Cellotetraose and cellopentose were hydrolyzed with all the enzymes to a high extent. Reducing sugars were released from Sigmacell, acid-swollen cellulose and filter paper, although the activity was only 0.2 to 4.0% of that shown against CMC. Since all enzymes are much more active on beta-glucans, carboxymethyl and hydroxyethyl cellulose, which gives more adjacent glucosyl residues in the cellulose backbone comparing with Sigmacell or filter paper, it is considered as more appropriate to refer to these enzymes as endo-beta-1,4-glucanases. This agrees also with the unability to hydrolyze soluble p-nitrophenyl derivatives. Xylanase activity was found in cell extracts expressing pBKRR.1 and pBKRR.14, although the activity was 20-40 times lower than that found with CMC.

In summary, it is concluded that: i) pBKRR1 and pBKRR14 have hallmark qualities of an endoglucanase which act mainly on beta-1,4-glucans but are also capable of hydrolyzing xylan, ii) pBKRR16 have both endoglucanase and cellobiohydrolase activity, and iii) pBKRR.3, 4, 7, 9, 11, 13, 19 and 22 are endo-beta-1,4-glucanases.

FIG. 2 represents Table 2 which contains the data obtained by purification of endoglucanases of seven clones (pBKRR.1, 9, 11, 13, 14, 16 and 22) with respect to the specific activity. Said clones were chosen, because they exhibit highest activity on the cellulose substrates. They were purified to homogeneity and their specific activity was estimated at pH 5.6 and 50° C., using CMC as substrate.

The specific activity is expressed in μmol min⁻¹ mg⁻¹ and gives the following values: 40.56 (μmol min⁻¹ mg⁻¹) for pBKRR.1, 41.59 for pBKRR.9, 6.42 for pBKRR.11, 65.00 for pBKRR.13, 34.60 for pBKRR.14, 4.12 for pBKRR.16 and 68.30 for pBKRR.22.

The average value of endoglucanase activity of the tested enzymes according to the invention was 4.5, 14, 2, 1.1 and 19 times higher than the activity of endoglucanases known in the art and isolated from hyperthermophilic archaeon Pyrococcus furiosus (Bauer et al., 1999), anaerobic fungus Orpinomyces joyonii strain SG4 (Qiu et al., 2000), two Bacillus strains, CH43 and HR68 (Mawadza et al., 2000), thermophilic fungus Thermoascus aurantiacus (Parry et al., 2002) and filamentous fungus Aspergillus niger (Hasper et al., 2002), respectively.

FIG. 3 represents Table 3 giving an overview over the biochemical properties of the cellulases according to the invention. Reaction was performed at the optimum pH and temperature for each done using CMC as substrate. The pH and temperature used in the reactions are shown in FIGS. 4 a-c. In detail, cell lysates from clones possessing higher activity endoglucanases were tested for their ability to hydrolyze CMC under pH conditions ranging from 3.5 to 10.5 at a temperature of 40° C. The values for clones pBKRR.1, 9, 11, 13, 14, 16 and 22 are depicted. For each clone the following data are given:
maximum pH at which the remaining activity after 24 h incubation at the indicate pH is >90% (column “Stable at pH”). It can be seen that pBKRR.22 showed the greatest activity-pH profile and showed activity at both alkaline (pH 9.0-10.0) and acidophilic (3.5-4.0) conditions without variation of enzyme activity. pBKRR.1 and pBKRR.9 were more active at pH 5.6, although they retained 65-80% of their activity between 3.5 and 7.0. pBKRR.11 showed maximal activity at pH 4.5 and its activity decreased at lower or higher pH. pBKRR.13 was active and stable at pH from 4.5 to 6.0 and pBKRR.14 and pBKRR.16 were more active at 5.6-6.0 and lost >60% of their activity at pH values of >2.
maximum temperature at which the remaining activity after 2 h incubation is >90% (column “Stable at Temperature”). Thermal activity and thermostability was determined at the pH optima pBKRR.9, pBKRR.14 and pBKRR.2 exhibited improved activity and thermostability at 70° C., pBKRR.1 and pBKRR.16 at 60° C., whereas pBKRR.11 and pBKRR.13 were more active at 50° C., losing >60% of their activity at higher temperatures. All cellulases exhibited calcium-independent thermostability except pBKRR.13 which exhibited greater thermostability at 60-70° C. in the presence of 4-10 mM Ca²⁺ compared with that shown in absence of this cation (Top temperature: 50° C.).
specific activity in μmol min⁻¹ g⁻¹ of cytoplasmatic extracts of E. coli containing the cloned cellulase genes,
solvent resistance given by remaining activity in a mixture buffer: DMSO (4:1). Activity was measured after 2 h incubation and compared with a control reaction in buffer lacking dimethylsulfoxide,
detergent resistance given by the remaining activity in the corresponding buffer (optimum pH) containing the indicate detergent at a concentration for 0 to 3%. Activity was measured after 2 h incubation and compared with a control reaction in buffer lacking detergent. pBKRR.1 and pBKRR.13 were 150 and 214% more active and stable at pH 5.6 and 70° C. or pH 5.6 and 50° C., respectively, in the presence of Triton X-100 (1-3%) but not in the presence of SDS (1 mM). PBKRR.19 was 184% and 108% more active and stable by 1-3% Triton X-100 and SDS at pH 4.5 and 70° C. Similar result was obtained for pBKRR.14 which was 134 and 161% more active in the presence of Triton X-100 and SDS (1-3%). PBKRR.22 was resistant to Triton X-100 up to 1-3% but was inhibited by SDS at concentrations higher than 1 mM. Only pBKRR.11 and pBKRR.16 showed medium stability in the presence of both surfactants (62-50% of the maximal activity). Other surfactants, such as Tween 20-80, polyoxyethylene alkyl ether or alkyl sulphate, -sulfonate, did not affect the activity of all the endoglucanases up to 3% w/v.
cation dependence: all indicated cations were added as chloride salts and tested at a concentration of 10 to 100 mM. As described above, all cellulases exhibited calcium independent thermostability except pBKRR.13. The effect of mono- (Na⁺, K⁺, NH4⁺, and divalent cations (Ca²⁺, Mn²⁺, Mg²⁺, Sr²⁺, Fe²⁺, Cu²⁺, Ni²⁺, Co²⁺, Zn²⁺) was first examined by treating the enzyme with chelating agents (EDTA and EGTA) to remove any endogenous divalent cations, followed by adding cations to the reaction mixture.

The results derived from these experiments showed that pBKRR.1, 11, 14 and 16 were not influenced by the tested cations, although Mg²⁺ showed a moderate stimulatory effect (127%). PBKRR.9 was slightly activated by all tested mono- and divalent cations (102 to 294%), whereas NH₄⁺ and Mg²⁺ partially inhibited the cellulases (10-15%). Interestingly, pBKRR.13 was highly activated (from 193 to 569%) by the majority of mono- and divalent cations including calcium (234%) which was the only done affected by this cation.

FIG. 4 a-c represent the results of the determination of pH and temperature optima showing graphically views of the pH-dependence activity and temperature-dependence activity of the polypeptides according to the invention. The pH optima of rumial endoglucanases were measured under conditions described in Example 5: 4% CMC, 0.5 mL of 50 mM buffer at the corresponding pH and 0.1 mg E. coli cell lysates, for 5-30 min, at 40° C. Effect of temperature on endoglucanase activity was measured in 50 mM sodium acetate buffer pH 5.6.

FIG. 4a shows a graphically view of the pH-dependent activity,

FIG. 4b shows a graphically view of the temperature-dependence activity and

FIG. 4c shows a graphically view of a comparison of the pH- and temperature-dependent activity at the same conditions. The percentage of the maximal response at each pH and temperature is given as follows: clones of the left side figures: pBKRR.1→pBKRR.9→pBKRR.11→pBKRR.13 (from the top to the bottom); right side figures pBKRR.14→pBKRR.16→pBKRR.22 (from the top to the bottom).

FIG. 5 corresponds to Table 4 exhibiting kinetic parameters (substrate saturation kinetics) for the hydrolysis of CMC by rumial endoglucanases. Data were calculated at optimal conditions of pH, temperature and Ca²⁺ requirements using CMC as substrate under conditions described in Example 5. The results of clones pBKRR.1, 9, 11, 13, 14, 16 and 22 are given. The saturation curve was hyperbolic (not shown), and the double-reciprocal plot of Lineweaver and Burke (1934) yielded an apparent kcat and Km as shown. Endoglucanases derived from pBKRR.1, pBKRR.9, pBKRR.13, pBKRR.14 and pBKRR.22 had a relatively low Km for CMC (from 0.160 to 0.259 mM) and quite high maximum catalytic efficiency (kcat/Km), which is in the range from 240 to 4710 s⁻¹ mM⁻¹. These results confirm that cellulases according to the invention have the highest catalytic efficiency towards CMC yet reported for any endoglucanase, and confirm the high affinity of endo-beta-1,4-glucanases isolated from rumen samples derived from New Zealand dairy cows for cellulose-based substrates.

FIG. 6 shows an comparison of specific activity of rumen cellulases with commercial counterparts. The activity is expressed in μmol min⁻¹ g⁻¹ of protein. The release of 1.0 μmol glucose from cellulose or CMC per minute at optimum pH corresponds to 1 unit. The results of rumen cellulases of clones pBKRR.1, 9, 11, 13, 14, 16 and 22 according to the invention are given. Results of experiments with commercially available cellulases from Aspergillus and Trichoderma are presented for comparative reasons.

To summerize the results shown in FIG. 6, the specific activity as well as the optimum pH and temperature of rumen cellulases are considerably higher than of the commercial cellulases. pBKRR.9 gives unprecedented results with a specific activity of 41.59 μmol min⁻¹ g⁻¹ at pH 7.0 and 70° C. and pBKRR.13 with a specific activity of 45.00 μmol min⁻¹ g⁻¹ at pH 7.0 and 50° C. followed by pBKRR.1, pBKRR.22 and pBKRR.14. All tested clones of the invention show much better results than the commercial enzymes (best result: a specific activity of 40.00 μmol min⁻¹ g⁻¹ at pH 4.5 and 45° C.).

FIG. 7 depicts the result of thin layer chromatography TLC analysis of products of CMC hydrolyzed by endoglucanases derived from a rumial genomic DNA library. The methods used for cellulase and substrate preparation, hydrolysis, TLC, and visualization are described in Example 5. The hydrolysis products glucose, cellobiose, cellotriose are shown an the right side. The amound of end products of CMC hydrolysis by the most active cellulases pBKRR.1, 9, 11, 13, 14, 16, 22 were determined by TLC. The main hydrolysis products were cellobiose and cellotriose. Only small amounts of glucose were detected using cells expressing pBKRR.16. Different pattern was found when using short-chain beta-1,4-glucans. Thus, the presence of cellobiose and traces of glucose were observed during hydrolysis of cellotriose only in the presence of cells expressing pBKRR.16. Cellotetraose was cleaved predominantly to cellobiose, whereas cellopentose was mainly hydrolyzed to cellobiose and cellotriose (not shown). Since small amounts of glucose were observed when using cells expressing pBKRR.16 for cellotetraose and cellopentaose, pBKRR.16 is able to hydrolize cellobiose and cellotriose.

FIGS. 9 to 16 represent the nucleic acid sequences of sixteen positive clones obtained from the genomic library constructed from ruminal ecosystem. In detail

FIG. 9 represents the identical nucleic acid sequence of clones pBKRR 1 and pBKRR 21. This clones contain two cellulases (endoglucanases) so that two nucleic acid sequences are represented in FIG. 9, namely RR01-1 (=RR21) and RR01-2 (=RR21).

FIG. 10 represents the identical nucleic acid sequence of clones pBKRR 2 and pBKRR 16. This clones contain two cellulases (endoglucanases) so that two nucleic acid sequences are represented in FIG. 10, namely RR02-1 (=RR16) and RR02-2 (=RR16).

FIG. 11 represents the identical nucleic acid sequence of pBKRR 22, pBKRR 6, pBKRR 8 and pBKRR 23.

FIG. 12 represents the nucleic acid sequence of pBKRR 7.

FIG. 13 represents the nucleic acid sequence of pBKRR 9.

FIG. 14 represents the identical nucleic acid sequence of pBKRR 11, pBKRR 10 and pBKRR 12.

FIG. 15 represents the nucleic acid sequence of pBKRR 13 and

FIG. 16 represents the identical nucleic acid sequence of pBKRR 14 and pBKRR 20-1.

FIGS. 17 to 24 represent the amino acid sequences corresponding to the nucleic acid sequences of the sit positive clones represented in FIGS. 9 to 16. In detail

FIG. 17 represents the amino acid sequence of pBKRR 1 and pBKRR 21. This clones contain two cellulases (endoglucanases) so that two amino acid sequences are represented in FIG. 17, namely RR01-1 (=RR21) and RR01-2 (=RR21).

FIG. 18 represents the amino acid sequence of pBKRR 2 and pBKRR 16. This clones contain two cellulases (endoglucanases) so that two amino acid sequences are represented in FIG. 18, namely RR02-1 (=RR16) and RR02-2 (=RR16).

FIG. 19 represents the amino acid sequence of pBKRR 22, pBKRR 6, pBKRR 8 and pBKRR 23.

FIG. 20 represents the amino acid sequence of pBKRR 7 polypeptide and mature protein).

FIG. 21 represents the amino acid sequence of pBKRR 9.

FIG. 22 represents the amino acid sequence of pBKRR 11, pBKRR 10 and pBKRR 12.

FIG. 23 represents the amino acid sequence of pBKRR 13 and

FIG. 24 represents the amino acid sequence of pBKRR 14 and pBKRR 20-1.

### EXAMPLES

### Materials and Buffers

Ostazin brilliant red-hydroxyethyl cellulose, potassium sodium tartrate, 3,5 dinitro-salicylic acid, carboxymethyl cellulose and Sigmacell® (type 101) were purchased from Sigma Chemical Co. (St Louis, Mo., USA). Hydroxyethyl-cellulose (medium viscosity), cellobiose, cellotriose, cellotetraose, cellopentaose and cellohexaose were purchased from Fluka (Oakville, ON).

Molecular mass markers for SDS- and native-PAGE were provided from Novagen and Amersham Pharmacia Biotech (Little Chalfont, United Kingdom), respectively. Restriction and modifying enzymes were obtained from New England Biolabs. DNase I grade II, was obtained from Boehringer Mannheim.

All other chemicals were of the highest grade commercially available. Unlike otherwise indicated, the standard buffer described here was 50 mM sodium acetate, pH 5.6.

### Example 1

### Production of a Genomic DNA Library from Rumen Ecosystem

A genomic DNA library from DNA purified directly from rumen ecosystem was generated as described previously (Ferrer et al., Molecular Biology (2004) 53 (1), pp. 167-182). Parallel to this the genomic DNA library was generated using the Stratagene Lambda ZAP Express Kit (Stratagene protocols, Catalog #239212, Catalog #239615 and Revision #053007) according to the manufacturers standard protocol.

### Example 2

### Endoglucanase Discovery and Expression Screening

Approximately 50,000 clones from selected genomic DNA libraries were plated to produce plaques on semi-solid medium according to standard procedures (Sambrook J, Maniatis T (1989) Molecular Cloning, A laboratory manual, Cold Spring Harbor Laboratory Press, Cold Spring Harbor, N.Y.). The library was chosen for endoglucanase discovery as follows: 25 ml of a solution 1% (w/v) Ostazin brilliant red-hydroxyethyl cellulose in NZY medium (sterilized by filtration in 0.22 μm filter disk) was added to 25 ml NZY soft-agar containing infected E. coli cells, after which the mixture was plated. The plates (25.5×22.5 cm) contained about 10000 phage clones, were incubated overnight (approx. 16 h). Positive plaques, identified by the appearance of a clearing zone or “halo”, were then purified. From the selected phages, the pBK-CMV plasmids have been excised using co-infection with helper phage, f1 and transferred to E. coli XLOLR (according to Strategene protocols, Catalog #239212, Catalog #239615 and Revision #053007). The cellulose encoding inserts were sequenced from both ends using universal primers T3 and T7. The inserts were sequenced furthermore by “primer walking approach” to sequence both strands of the insert completely.

### Example 3

### Subcloning and Expression of Endoglucanase Genes

Endoglucanase genes from pBK-CMV plasmids obtained previously (Example 2) were expressed in E. coli XLOLR that were grown to mid-log phase and induced with 0.1 mM isopropyl-β-D-galactopyranoside for 2 h at 37° C. Cell growth rate was measured by absorbance at 600 nm using a Beckman DU-7400 spectrophotometer. Cells were resuspended in standard buffer and subjected to lyophilization. The subcloning and expression of the endoglucanase genes were performed according to the Stratagene protocols, Catalog #239212, Catalog #239615 and Revision #053007 (according to the manufacturers standard protocol).

### Example 4

### Recovery of Endoglucanases

The endoglucanases were recovered from 1-liter shaked flask fermentations, in Luria-Bertany broth plus 50 μg of Kanamycin per ml. One gram of fermentation broth was mixed with 10 ml of standard buffer in a 50-ml Falcon tube. The solution was vortexed and then incubated with DNase I grade II, for 30-45 min, and then sonicated for 4 min total time. A sample of the soluble fraction was separated from insoluble debris by centrifugation (10,000×g, 30 min, 4° C.) and proteins were precipitated by addition of 80% chilled acetone (−20° C.). Proteins were resuspended in standard buffer and stored at −20° C. at a concentration of 5 mg/ml until use. The amount of protein expression was examined using SDS-PAGE with 10-12% acrylamide. Gels were stained with Coomasie Blue and the appropriate molecular weight region was examined for determination of endoglucanase protein compared with the total protein. Cellulases were purified by preparative non-denaturing PAGE (5-15% polyamide) at 45 V constant power at 4° C. according to the manufacturer's (Bio-Rad) protocol. The gel region containing the active cellulase, detected in a parallel track by activity staining (using Ostazin brillian-blue carboxymethyl cellulose) was excised, suspended in two volumes of standard fuffer and homogenized in a glass tissue homogenizer. The eluate obtained after removal of polyacrilamide by centrifugation at 4,500 g at 4° C. for 15 min was concentrated by ultrafiltration on a Centricon YM-10 (Amicon, Millipore) to a total volume of 1000 μl. The sample was further purified on a Superose 12 HR 10/30 gel filtration column pre-equilibrated with standard buffer containing 150 mM NaCl. Separation was performed at 4° C. at a flow rate of 0.5 ml/min.

### Example 5

### Cellulase Activity Assay

The standard cellulase activity was determined in a continuous spectrophotometric assay by measuring the release of reducing sugars from the 4% (wt/vol carboxymethyl cellulose (CMC) in 0.5-ml reaction mixtures containing 50 mM sodium acetate buffer, pH 5.6, at 40° C., using the dinitrosalycilic acid (DNS) method (Bernfeld, P. (1955) Amilases and b. In: Colowick S P, Kaplan N O (eds). Methods in Enzymology, Academic Press, New York pp. 133-155). The reaction was performed in 0.5 ml scale containing 20 mg substrate and 5 mg lyophilised cells or 0.1 mg purified enzyme. Reactions were allow to proceed for a time period of 5-30 min after which the samples were centrifuge (10,000×g, 3 min). For determination of reducing sugars, 50 μl of each sample was taken and mixed with 50 μl of dinitrosalycilic acid (DNS) solution (0.2 g DNS+4 ml NaOH (0.8 g/10 ml)+10 ml H2O+6 g potassium sodium tartrate) in 96-well microtiter plates. The samples were heated at 95° C. for 30 min and cooled to room temperature. Finally, each well was diluted with 150 μl water and the absorbance at 550 nm was measured. Hydrolytic activity towards others cellulose substrates, i.e. hydroxyethylcellulose, Sigmacell and acid-swollen cellulose, laminarin and barley glucan was performed using conditions described for the standard cellulose assay, as described above. Reaction mixtures were mixed by mechanical agitation at 1,000 rpm. All enzyme assays were determined to be linear with respect to time and protein concentration. Sample blanks were used to correct for non-enzymatic release of the reduced sugar. One unit is defined as the amount of enzyme liberating 1 μmol of glucose-equivalent reducing groups per minute. Non-enzymatic hydrolysis of the substrates at elevated temperatures was corrected for with the appropriate blanks.

The products formed by hydrolysis of cellulose-based substrates (cellobiose, cellotetraose, cellopentaose, cellulose and carboxymethyl cellulose) were analysed by thin-layer chromatography (TLC). Solutions containing 20 μg of E. coli cell lysate proteins and 0.2% (wt/vol) substrate in 50 mM acetate buffer (pH 5.6) were incubated at 50° C. until maximum conversion was achieved. The reactions were terminated by heating the preparation for 5 min at 95° C. and hydrolysis products were separated by TLC on silica gel plates (Merck, Darmstadt, Germany) by using a mixture chloroform, glacial acetic acid, and water (6:14:1) as the solvent. Glucose, cellobiose and cellotriose were used as standard for the identification of the hydrolysis products. Sugars were visualized by heating 105° C. for 15 min the plates which were previously sprayed with a reagent containing amine (2 ml), diphenylamine (2 g), acetone (100 ml) and 85% H3PO4 (15 ml).

Hydrolytic activity using p-nitrophenyl derivatives as substrates was assayed spectrophotometrically at 405 nm, in 96-well microtiter plates. Briefly, the enzyme activity was assayed by the addition of 5 μl enzyme solution (50 μM) to 5 μl of 32 mm p-nitrophenyl glycosides (Sigma) stock solution (in acetonitrile), in 2.850 μl 50 mM sodium acetate buffer, pH 5.6. Reaction was allow to proceed from 2 to 300 min, at 40° C. and the hydrolytic reaction was monitored. One unit of enzymatic activity was defined as the amount of protein releasing 1 μmol of p-nitrophenoxide/min from nitrophenyl glycoside at the indicated temperature and pH.

The optimal pH for enzyme activity was measured by incubating the enzyme substrate mixture at pH values ranging from 5.5 to 10.5 for 5-30 min at 40° C., using the standard method described above and CMC as substrate. The different buffers used were sodium citrate (pH 3.5-4.5), sodium acetate (pH 5.0-6.0), HEPES (pH 7.0), Tris-HCl (pH 8.0-9.0) and glycine-NaOH (pH 9.0-10.5), at a concentration of 50 mM. For temperature-dependent assay 50 mM sodium acetate buffer pH 5.6, was chosen. The optimal temperature for activity was determined quantitatively after incubating the enzyme substrate (CMC) mixture at temperatures ranging from 4 to 80° C. The solution was allowed to proceed for 3-30 min after which the enzymatic activity was measured as described above. In a second set of experiments, the pH and thermal stability was determined in the presence (40 g/l) or absence of calcium by preincubating the enzyme at pH from 3.5 to 10.5 and temperatures ranging from 30 to 80° C. 5 μl aliquots were withdrawn at times and remaining cellulase activity was measured at 40° C. using the standard assay as described above. Activity pre- and postincubation was measured to calculate residual activity.

### Example 6

### Effect of Various Chemicals on Cellulase Activity

To study the effect of cations, inhibitors and surfactants on endoglucanase activity, the conditions were as follows: All cations tested were added as chloride salts and tested at a concentration of 10 mM. Inhibitors such as N-ethylmalemide, iodoacetate and p-chloromercuribenzoate at concentration from 1-5 mM were incubated in the presence of the enzyme for 30 min (30° C.) prior to the addition of the substrate (CMC). The effects of detergents on the endoglucanase activity was analysed by adding of 1-3% (wt/vol) detergent to the enzyme solution. In all cases, the enzyme activity was assayed in triplicate using the standard cellulose assay described above and the residual activity was expressed as percent of the control value (without addition of chemicals).

### Example 7

### Protein Determination and N-Terminal and Internal Amino Acid Sequence

Concentrations of soluble proteins were determined by the Bradford dye-binding method with a Bio-Rad Protein Assay Kit with bovine serum albumin as standard (Bradford, M M (1976) A rapid and sensitive method for the quantification of microgram quantities of protein utilizing the principle of protein-dye binding, Anal Biochem 72:248-254.). SDS-PAGE and native electrophoresis were performed according to Laemmli, UK (1970), Cleavage of structural proteins during the assembly of the head of bacteriophage T4, Nature 227:680-685. For NH₂-terminal amino acid sequencing, the purified proteins were subjected to PAGE in the presence of sodium dodecyl sulfate (SDS) and protein bands were blotted to a polyvinylidene difluoride membrane (Millipore Corp.) using semidry blot transfer apparatus according to the manufacturer's instructions. The blotted membrane was stained with Coomassie Brilliant Blue R250, and after destaining with 40% methanol/10% acetic acid the bands were cut out and processed for N-terminal amino acid sequence. The internal sequence was determined as follows: after SDS-PAGE (10-15% acrylamide) the protein bands stained with Coomassie brilliant blue R-250 were cut out and digested with trypsin and then sequenced using a protein sequencer (Bruker Daltonics).

1-26. (canceled)

27. Polypeptide comprising one of the amino acid sequences of amino acids No. 175 to No. 210 of the amino acid sequences shown in FIGS. 17 to 24 or a functional fragment, or functional derivative thereof.

28. Polypeptide of claim 27, wherein the polypeptide comprises one of the amino acid sequences of amino acids No. 175 to 210, preferably No. 170 to No. 220, more preferably No. 150 to No. 240, most preferably No. 120 to 280 of the amino acid sequences, shown in FIGS. 17 to 24.

29. Polypeptide of claim 27, wherein the polypeptide comprises one of the amino acid sequences shown in FIGS. 17 to 24 or a functional fragment, or functional derivative thereof.

30. Polypeptide of claim 27, wherein the polypeptide is derived from rumen, particularly from rumen ecosystem, preferably from cow rumen, more preferably from New Zealand dairy cow.

31. Polypeptide of claim 27, wherein the polypeptide shows activity at pH optimum, preferably at pH ranging from 3.5 to 10.0, more preferably from 3.5 to 5.5 or from 5.5 to 7.0 or from 6.5 to 9.0, most preferably from 7.0 to 10.0.

32. Polypeptide of claim 27, wherein the polypeptide shows activity at temperature optimum, preferably at a temperature from 40° C. to 70° C., more preferably 40° C. to 60° C., most preferably from 50° C. to 70° C.

33. Polypeptide of claim 27, wherein the polypeptide shows activity at low addition of cations, preferably without any addition of cations.

34. Polypeptide of claim 27, wherein the polypeptide shows high specific activity towards its substrate.

35. Polypeptide of claim 27, wherein the polypeptide shows high stability towards its substrate.

36. Polypeptide of claim 27, wherein the polypeptide shows a combination of at least two features, preferably three features, more preferably four features, most preferably five features, selected from:
a. being derived from rumen, particularly from rumen ecosystem, preferably from cow rumen, more preferably from New Zealand dairy cow;
b. showing activity at pH optimum, preferably at pH ranging from 3.5 to 10.0, more preferably from 3.5 to 5.5 or from 5.5 to 7.0 or from 6.5 to 9.0, most preferably from 7.0 to 10.0;
c. showing activity at temperature optimum, preferably at a temperature from 40° C. to 70° C., more preferably 40° C. to 60° C., most preferably from 50° C. to 70° C.;
d. showing activity at low addition of cations, preferably without any addition of cations;
e. showing high specific activity towards its substrate.

37. A nucleic acid encoding a polypeptide of claim 27 or a functional fragment or functional derivative thereof.

38. The nucleic acid of claim 37 comprising or consisting of one of the nucleic acid sequences of FIG. 9 to 16.

39. A vector comprising the nucleic acid of claim 36.

40. A host cell comprising the vector of claim 38.

41. A method for the production of the polypeptide of claim 27 comprising the following steps:
a. cultivating a host cell, said host cell comprising a nucleic acid encoding said polypeptide and expressing the nucleic acid under suitable conditions; and
b. isolating the polypeptide with suitable means.

42. An ingredient for a composition for treating cellulosic textiles or fabrics, said ingredient comprising the polypeptide of claim 27, a nucleic acid enclosing said polypeptide, functional fragments or functional derivatives thereof.

43. An ingredient for a consumer product such as food product, or an animal food product, said ingredient comprising the polypeptide of claim 27, a nucleic acid enclosing said polypeptide, functional fragments or functional derivatives thereof.

44. A method for treating paper pulp comprising contacting said pulp with the polypeptide of claim 27 or a nucleic acid encoding said polypeptide, functional fragment, or functional derivatives thereof.

45. An ingredient for a protoplast preparation agent, a herbicide or a drench in ruminants, said ingredient comprising the polypeptide of claim 27, a nucleic acid enclosing said polypeptide, functional fragments or functional derivatives thereof.

46. A method for producing anti-cellulase reagents comprising contacting said pulp with the polypeptide of claim 27 or a nucleic acid encoding said polypeptide, functional fragments, or functional derivatives thereof.
